(19) United States
(12) Patent Application Publication
(10) Pub. No.: US 2005/0220723 A1
(43) Pub. Date: Oct. 6, 2005

(54) Aerosol device comprising a propellant and a styling composition comprising, in a predominantly aqueous medium, a pseudo-block polymer and an additional fixing polymer; processes therefor and uses thereof

(76) Inventors: Katarina Benabdillah, Clichy (FR); Jonathan Gawtrey, Boulogne (FR)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/089,172

(22) Filed: Mar. 25, 2005

Related U.S. Application Data
(60) Provisional application No. 60/572,106, filed on May 19, 2004.

(30) Foreign Application Priority Data
Mar. 25, 2004 (FR) 0403088

(51) Int. Cl. A61K 9/00

(52) U.S. Cl. 424/47

(57) ABSTRACT

An aerosol device comprising at least one propellant and at least one styling cosmetic composition comprising, in a cosmetically acceptable medium predominantly comprising water, at least one pseudo-block polymer and at least one additional fixing polymer; cosmetic treatment process for shaping and/or holding the hairstyle, comprising vaporizing onto the hair the contents of the aerosol device, and also to the uses of the contents of the aerosol devices for obtaining fixing of the hair that is persistent over time and/or has good moisture resistance and/or good cosmeticity.

This application claims benefit of U.S. Provisional Application No. 60/572,106, filed May 19, 2004, the contents of which are incorporated herein by reference. This application also claims benefit of priority under 35 U.S.C. § 119 to French Patent Application No. FR 0403088, filed Mar. 25, 2004, the contents of which are also incorporated by reference.

Disclosed herein is an aerosol device comprising a propellant and a styling cosmetic composition comprising, in a cosmetically acceptable medium predominantly comprising water, at least one pseudo-block polymer and at least one additional fixing polymer.

The cosmetic compositions for shaping and/or holding the hairstyle that are the most widely available on the cosmetics market are spray compositions comprising a solution, usually an alcoholic solution, and at least one component, known as fixing components, which are generally random polymer resins whose function is to form welds between the hairs. These fixing components may often be formulated as a mixture with various cosmetic adjuvants. The cosmetic compositions may generally be packaged either in a suitable aerosol container pressurized using a propellant, or in a pump-dispenser bottle.

Aerosol systems for fixing hair may comprise a liquid phase (or fluid) and a propellant. The liquid phase may comprise the fixing components and a suitable solvent.

Once applied to the hair, the liquid phase dries, allowing the formation of welds required for the fixing of the hair by the fixing components. The welds should be rigid enough to hold the hairs, and should do so with a sufficient persistence of effects, such as moisture resistance. However, the welds should also be fragile enough for the user to be able, by combing or brushing the hair, to destroy them without injuring the scalp or damaging the hair. The cosmetic compositions should also be stable over time. At least one good cosmetic effect on the hair may also be sought, for example, softness and disentangling.

It is moreover sought to reduce the amount of volatile solvents present in these compositions, for environmental reasons. The total or partial replacement of the volatile solvents, such as alcohol, with water may generally be reflected by a reduction in fixing properties, in the persistence of the styling effect over time and in the cosmetic properties.

The present disclosure relates to a combination of at least one pseudo-block polymer and at least one fixing polymer which may make it possible to obtain predominantly aqueous styling compositions which, when introduced into an aerosol device, may make it possible to obtain good fixing and good hold of the hair, i.e. a styling effect that persists throughout the day, or even for several days, with good moisture resistance, and which is easy to remove by shampooing. These compositions may also make it possible to give the hair at least one good cosmetic property, such as softness or disentangling.

Furthermore, the compositions predominantly aqueous base makes it highly ecologically advantageous.

Disclosed herein is thus an aerosol device comprising at least one propellant and at least one styling composition comprising, in a cosmetically acceptable medium predominantly comprising water, at least one pseudo-block polymer and at least one additional fixing polymer.

Further disclosed herein is a cosmetic treatment process for shaping and/or holding the hairstyle, comprising vaporizing onto the hair of the contents of the disclosed aerosol device.

Even further disclosed herein are uses of the aerosol device disclosed herein to obtain, after vaporization of the contents onto a head of hair, fixing of the hair that is persistent over time and/or that has good moisture resistance and/or that has good cosmeticity.

Further disclosed herein is the use of the aerosol device disclosed herein to obtain a styling mousse.

Other subjects, characteristics, aspects and advantages of the embodiments disclosed herein will emerge even more clearly on reading the description and the examples that follow.

As used herein, the term “styling composition” means a composition for shaping and/or holding the hairstyle.

As used herein, the term “medium predominantly comprising water” means a medium comprising more than 50%, for example, more than 70% and, further, for example, more than 85% by weight of water, relative to the total weight of the at least one styling composition. In one embodiment, the medium comprises upwards of 99.9% by weight of water, relative to the total weight of the at least one styling composition.

The cosmetically acceptable medium is a medium comprising water and optionally at least one organic solvent.

As used herein, the term “organic solvent” means an organic compound with a molecular weight of less than 500, which is liquid at a temperature of 25° C. and at atmospheric pressure. In one embodiment, the organic compound is polar.

In one embodiment, the at least one organic solvent is an alcohol. For example, the at least organic solvent may be chosen from C₁-C₄ lower alcohols, such as ethanol, isopropanol, tert-butanol, and n-butanol; polyols, for example, propylene glycol, and polyol ethers, in one embodiment the alcohol is an ethanol.

The aerosol devices disclosed herein comprise at least one propellant. Any suitable propellant may be used. In some embodiments, the aerosol devices may be one-compartment devices, in which case the at least one propellant may be chosen, for example, from liquefied and compressed gases such as nitrogen, dimethyl ether and hydrocarbons. In some embodiments, the aerosol devices may also be two-compartment devices, for example, an aerosol device comprising a pocket with propulsion by air or with a compressed gas.

The at least one propellant may be present in an amount ranging from 6% to 70%, for example, from 6% to 50% and, further, for example, from 30% to 45% by weight, relative to the total weight of materials comprised in the aerosol device. The at least one styling composition in the aerosol device may be present in an amount ranging from 30% to 94%, for example, from 50% to 94% and, even further, for example, from 55% to 70% by weight, relative to the total weight of materials comprised in the device.

Pseudo-Block Polymer

The at least one pseudo-block polymer used in the at least one styling composition disclosed herein is chosen from block polymers comprising at least one first block and at least one second block that are mutually incompatible and that have different glass transition temperatures (Tg), wherein the at least one first and the at least one second block are linked together via an intermediate segment comprising at least one constituent monomer of the at least one first block and at least one constituent monomer of the at least one second block, and wherein the at least one pseudo-block polymer has a polydispersity index (I) of greater than 2.

As use herein, the term “at least” one block means one or more blocks.

As used herein, the term “mutually incompatible blocks” means that the mixture formed from the polymer corresponding to the at least one first block and the polymer corresponding to the at least one second block is immiscible in the polymerization solvent that is of the majority amount by weight of the at least one pseudo-block polymer, at room temperature (25° C.) and atmospheric pressure (10⁵ Pa), for a polymer mixture content of greater than or equal to 5% by weight, relative to the total weight of the mixture (polymers and solvent), wherein:
i) the polymers corresponding to the at least one first and at least one second blocks are present in the mixture in an amount such that the respective weight ratio ranges from 10:90 to 90:10 and
ii) each of the polymers corresponding to the at least one first and at least one second blocks has an average (weight-average or number-average) molecular mass equal to that of the at least one pseudo-block polymer±15%.

In the case wherein the polymerization solvent comprises a mixture of polymerization solvents, and in the event of two or more solvents present in identical mass proportions, the polymer mixture is immiscible in at least one of them.

Needless to say, in the case of a polymerization performed in only one solvent, this solvent is present in the majority amount.

The intermediate segment is a block comprising at least one constituent monomer of the at least one first block and at least one constituent monomer of the at least one second block of the polymer; it allows these blocks to be “compatibilized”.

In one embodiment, the at least one pseudo-block polymer disclosed herein may be chosen from film-forming linear block ethylenic polymers.

As used herein, the term “ethylenic” polymer means a polymer obtained by polymerization of ethylenically unsaturated monomers.

As used herein, the term “block” polymer means a polymer comprising at least two different blocks, such as at least three different blocks.

The at least one pseudo-block polymer is a polymer of linear structure. In contrast, a polymer of non-linear structure is, for example, a polymer of branched, star or grafted structure, or the like.

As used herein, the term “film-forming” polymer means a polymer that is capable of forming by itself or in the presence of an auxiliary film-forming agent, a continuous film that adheres to a support, such as to keratin materials.

In some embodiments, the at least one pseudo-block polymer disclosed herein does not comprise any silicon atoms in its skeleton. As used herein, the term “skeleton” means the main chain of the polymer, as opposed to the pendent side chains.

In one embodiment, the at least one pseudo-block polymer disclosed herein is not water-soluble, i.e. the polymer is not soluble in water or mixtures of water and linear or branched lower monoalcohols comprising from 2 to 5 carbon atoms, such as ethanol, isopropanol or n-propanol, without pH modification, at an active material content of at least 1% by weight, at room temperature (25° C.).

In one embodiment, the at least one pseudo-block polymer disclosed herein is not an elastomer.

As used herein, the term “non-elastomeric polymer” means a polymer which, when it is subjected to a constraint intended to stretch it (for example by 30% relative to its initial length), does not return to a length substantially identical to its initial length when the constraint ceases.

More specifically, the term “non-elastomeric polymer” means a polymer with an instantaneous recovery Rᵢ<50% and a delayed recovery R₂ₕ<70% after having been subjected to a 30% elongation. For example, Rᵢ is <30% and R₂ₕ<50%.

More specifically, the non-elastomeric nature of the at least one pseudo-block polymer used is determined according to the following protocol:

A polymer film is prepared by pouring a solution of the polymer in a Teflon-coated mould, followed by drying for 7 days in an environment conditioned at 23±5° C. and 50±10% relative humidity.

A film on the order of 100 μm thick is thus obtained, from which are cut rectangular specimens (for example using a punch) 15 mm wide and 80 mm long.

This sample is subjected to a tensile stress using a machine sold under the reference Zwick, under the same temperature and humidity conditions as for the drying.

The specimens are pulled at a speed of 50 mm/min and the distance between the jaws is 50 mm, which corresponds to the initial length (l₀) of the specimen.

The instantaneous recovery Rᵢ is determined in the following manner:
the specimen is pulled by 30% (εₘₐₓ), i.e. on the order of 0.3 times its initial length (l₀)
the constraint is released by applying a return speed equal to the tensile speed, i.e. 50 mm/min, and the residual elongation of the specimen is measured as a percentage, after returning to zero constraint (εᵢ).

The percentage instantaneous recovery (Rᵢ) is given by the following formula:
Rᵢ=(εₘₐₓ−εᵢ)/εₘₐₓ)×100

To determine the delayed recovery, the percentage residual elongation of the specimen (ε₂ₕ) is measured, 2 hours after returning to zero constraint.

The percentage delayed recovery (R₂ₕ) is given by the following formula:
R₂ₕ=(εₘₐₓ−ε₂ₕ)/εₘₐₓ)×100

Purely as a guide, a polymer according to one embodiment has an instantaneous recovery Rᵢ of 10% and a delayed recovery R₂ₕ of 30%.

For example, the at least one pseudo-block polymer used in the compositions disclosed herein comprises at least one first block and at least one second block that are mutually incompatible and that have different glass transition temperatures (Tg), wherein the at least one first and the at least one second block are linked together via an intermediate segment comprising at least one constituent monomer of the at least one first block and at least one constituent monomer of the at least one second block, wherein the at least one pseudo-block polymer has a polydispersity index (I) of greater than 2.

It is pointed out that, in the text hereinabove and hereinbelow, the terms “first block” and “second block” do not in any way condition the order of the blocks in the structure of the at least one pseudo-block polymer.

The polydispersity index (I) of the at least one pseudo-block polymer is equal to the ratio of the weight-average mass (Mw) to the number-average mass (Mn).

The weight-average molar mass (Mw) and number-average molar mass (Mn) are determined by gel permeation liquid chromatography (THF solvent, calibration curve established with linear polystyrene standards, refractometrc detector).

The weight-average mass (Mw) of the at least one pseudo-block poly mer used in the composition disclosed herein is, for example, less than or equal to 300,000; it ranges, for example, from 35,000 to 200,000, such as from 45,000 to 150,000.

The number-average mass (Mn) of the at least one pseudo-block polymer used in the composition disclosed herein is, for example, less than or equal to 70,000; it ranges, for example, from 10,000 to 60,000, such as from 12,000 to 50,000.

In one embodiment, the polydispersity index of the at least one pseudo-block polymer used in the at least one styling composition disclosed herein is greater than or equal to 2, for example, ranging from 2 to 9, such as greater than or equal to 2.5, for example, ranging from 2.5 to 8, such as greater than or equal to 2.8, and, for example, ranging from 2.8 to 6.

Each block of the at least one pseudo-block polymer used in the at least one styling composition disclosed herein is derived from one type of monomer or from several different types of monomers.

This means that each block may comprise a homopolymer or a copolymer; this copolymer constituting the block may in turn be random or alternating.

In one embodiment, the intermediate segment comprising at least one constituent monomer of the at least one first block and at least one constituent monomer of the at least one second block of the at least one pseudo-block polymer is a random polymer.

In another embodiment, the intermediate segment is derived essentially from constituent monomers of the at least one first block and of the at least one second block.

As used herein, the term “essentially” means at least 85%, such as at least 90%, for example, at least 95% and, further, for example, 100%.

In another embodiment, the intermediate segment has a glass transition temperature Tg that is between the glass transition temperatures of the at least one first and at least one second blocks.

The at least one first and at least one second blocks have different glass transition temperatures.

The glass transition temperatures indicated for the at least one first and at least one second blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:

$$1 / Tg = {\sum }_{i} ( {ϖ}_{i} / {Tg}_{i} ) ,$$

Wherein: \overline{ω}ᵢ is the mass fraction of the monomer (i) in the block under consideration and Tgᵢ is the glass transition temperature of the homopolymer of the monomer (i).

Unless otherwise indicated, the Tg values indicated for the at least one first and at least one second blocks disclosed herein are theoretical Tg values.

The difference between the glass transition temperatures of the at least one first and at least one second blocks may be greater than 10° C., for example, greater than 20° C. and, further, for example, greater than 30° C.

In some embodiments, the at least one first block may be chosen from:
a) a block with a Tg of greater than or equal to 40° C.,
b) a block with a Tg of less than or equal to 20° C.,
c) a block with a Tg ranging from 20 to 40° C.,
and the at least one second block may be chosen from a category a), b) or c) different from the at least one first block.

a) Block with a Tg of Greater than or Equal to 40° C.

The block with a Tg of greater than or equal to 40° C. has, for example, a Tg ranging from 40 to 150° C., such as greater than or equal to 50° C., for example, ranging from 50° C. to 120° C., and, further, such as from 50° C. to 100° C. and, further, such as greater than or equal to 60° C., for example, ranging from 60° C. to 120° C.

The block with a Tg of greater than or equal to 40° C. may be chosen from homopolymers and copolymers.

In the case where this block is a homopolymer, it is derived from at least one monomer, which is such that the homopolymer prepared from the at least one monomer has a glass transition temperature of greater than or equal to 40° C. This first block may be a homopolymer comprising only one type of monomer (for which the Tg of the corresponding homopolymer is greater than or equal to 40° C.).

In the case where the first block is a copolymer, it may be totally or partially derived from at least one monomer, the nature and concentration of which are chosen such that the Tg of the resulting copolymer is greater than or equal to 40° C. The copolymer may comprise, for example:
monomers which are such that the homopolymers prepared from these monomers have Tg values of greater than or equal to 40° C., for example, a Tg ranging from 40 to 150° C., such as greater than or equal to 50° C., for example, ranging from 50° C. to 120° C. and, further, such as greater than or equal to 60° C., for example, ranging from 60° C. to 120° C., and
monomers which are such that the homopolymers prepared from these monomers have Tg values of less than 40° C., chosen from monomers with a Tg ranging from 20 to 40° C. and monomers with a Tg of less than or equal to 20° C., for example, a Tg ranging from −100 to 20° C., such as less than 15° C., further, for example, ranging from −80° C. to 15° C. and, further, such as less than 10° C., for example, ranging from −50° C. to 0° C., as described later.

The at least one monomer whose homopolymer has a glass transition temperature of greater than or equal to 40° C. is chosen, for example, from the following monomers, also known as main monomers:
methacrylates of formula CH₂═C(CH₃)—COOR₁
wherein R₁ is chosen from linear and branched unsubstituted alkyl groups comprising from 1 to 4 carbon atoms, such as a methyl, ethyl, propyl, and isobutyl groups or R₁ is chosen from C₄ to C₁₂ cycloalkyl groups;
acrylates of formula CH₂═CH—COOR₂
wherein R₂ is chosen from C₄ to C₁₂ cycloalkyl groups, such as an isobornyl group and a tert-butyl group; and
(meth)acrylamides of formula: 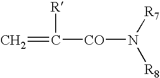
wherein R₇ and R₈, which may be identical or different, are each chosen from a hydrogen atom and linear and branched C₁ to C₁₂ alkyl groups, such as n-butyl, t-butyl, isopropyl, isohexyl, isooctyl, and isononyl groups; or R₇ is H and R₈ is a 1,1-dimethyl-3-oxobutyl group,
   and R′ is chosen from H and methyl. Examples of monomers that may be mentioned include N-butylacrylamide, N-t-butylacrylamide, N-isopropylacrylamide, N,N-dimethylacrylamide, and N,N-dibutylacrylamide.

In some embodiments, the at least one monomer whose homopolymer has a glass transition temperature of greater than or equal to 40° C. may be chosen from methyl methacrylate, isobutyl(meth)acrylate and isobornyl(meth)acrylate.

b) Block with a Tg of Less than or Equal to 20° C.

The block with a Tg of less than or equal to 20° C. has, for example, a Tg ranging from −100 to 20° C., for example, less than or equal to 15° C., such as ranging from −80° C. to 15° C. and, further, for example, less than or equal to 10° C., for example, ranging from −100° C. to 0° C., such as ranging from −50° C. to 0° C.

The block with a Tg of less than or equal to 20° C. may be chosen from homopolymer and copolymers.

In the case where this block is a homopolymer, it is derived from at least one monomer which is such that the homopolymer prepared from the at least one monomer has a glass transition temperature of less than or equal to 20° C. This second block may be a homopolymer comprising only one type of monomer (for which the Tg of the corresponding homopolymer is less than or equal to 20° C.).

In the case where the block with a Tg of less than or equal to 20° C. is a copolymer, it may be totally or partially derived from at least one monomer, the nature and concentration of which are chosen such that the Tg of the resulting copolymer is less than or equal to 20° C.

It may comprise, for example
at least one monomer whose corresponding homopolymer has a Tg of less than or equal to 20° C., for example, a Tg ranging from −100° C. to 20° C., such as less than 15° C., for example, ranging from −80° C. to 15° C. and, further, such as less than 10° C., for example, ranging from −50° C. to 0° C., and
at least one monomer whose corresponding homopolymer has a Tg of greater than 20° C., such as monomers with a Tg of greater than or equal to 40° C., for example, a Tg ranging from 40 to 150° C., such as greater than or equal to 50° C., for example, ranging from 50° C. to 120° C. and, further, for example, greater than or equal to 60° C., even further, for example, ranging from 60° C. to 120° C. and/or monomers with a Tg ranging from 20 to 40° C., as described above.

In some embodiments, the block with a Tg of less than or equal to 20° C. is a homopolymer.

The at least one monomer whose homopolymer has a Tg of less than or equal to 20° C. may, for example, be chosen from the following monomers, or main monomers:
acrylates of formula CH₂═CHCOOR₃,
wherein R₃ is chosen from linear and branched C₁ to C₁₂ unsubstituted alkyl groups, with the exception of the tert-butyl group, wherein at least one hetero atom chosen from O, N and S is optionally intercalated;
methacrylates of formula CH₂═C(CH₃)—COOR₄,
wherein R₄ is chosen from linear and branched C₆ to C₁₂ unsubstituted alkyl groups, wherein at least one hetero atom chosen from O, N and S is optionally intercalated;
vinyl esters of formula R₅—CO—O—CH═CH₂,
wherein R₅ is chosen from linear and branched C₄ to C₁₂ alkyl groups; and
C₄ to C₁₂ alkyl vinyl ethers,
N—(C₄ to C₁₂)alkyl acrylamides, such as N-octylacrylamide.

At least one of the monomers that may be used for the block with a Tg of less than or equal to 20° C. may be chosen from alkyl acrylates whose alkyl chain comprises from 1 to 10 carbon atoms, with the exception of the tert-butyl group, such as methyl acrylate, isobutyl acrylate, 2-ethylhexyl acrylate.

c) Block with a Tg Ranging from 20 to 40° C.

The block with a Tg ranging from 20 to 40° C. may be chosen from homopolymers and copolymers.

In the case where this block is a homopolymer, it is derived from at least one monomer (or main monomer) which is such that the homopolymer prepared from the at least one monomer has a glass transition temperature ranging from 20 to 40° C. This first block may be a homopolymer, comprising only one type of monomer (wherein the Tg of the corresponding homopolymer ranges from 20° C. to 40° C.).

The at least one monomer whose homopolymer has a glass transition temperature ranging from 20 to 40° C. may be chosen from n-butyl methacrylate, cyclodecyl acrylate, neopentyl acrylate and isodecylacrylamide.

In the case where the block with a Tg ranging from 20 to 40° C. is a copolymer, it is totally or partially derived from at least one monomer (or main monomer) whose nature and concentration is chosen such that the Tg of the resulting copolymer ranges from 20 to 40° C.

For example, the block with a Tg ranging from 20 to 40° C. may be chosen from copolymers totally or partially derived from:
main monomers whose corresponding homopolymer has a Tg of greater than or equal to 40° C., for example, a Tg ranging from 40° C. to 150° C., such as greater than or equal to 50° C., for example, ranging from 50 to 120° C. and, further, such as greater than or equal to 60° C., for example, ranging from 60° C. to 120° C., as described above, and
main monomers whose corresponding homopolymer has a Tg of less than or equal to 20° C., for example, a Tg ranging from −100 to 20° C., such as less than or equal to 15° C., for example, ranging from −80° C. to 15° C. and, further, such as less than or equal to 10° C., for example, ranging from −50° C. to 0° C., as described above,
wherein the monomers are chosen such that the Tg of the copolymer forming the first block ranges from 20 to 40° C.

The at least one monomer may be chosen, for example, from methyl methacrylate, isobornyl acrylate and methacrylate, butyl acrylate, triflouroethyl methacrylate, and 2-ethylhexyl acrylate.

In some embodiments, the proportion of the second block with a Tg of less than or equal to 20° C. ranges from 10% to 85% by weight, for example, from 20% to 70% and, even further, for example, from 20% to 50% by weight of the at least one pseudo-block polymer.

However, each of the blocks may contain in small proportion at least one constituent monomer of the other block.

Thus, the at least one first block may contain at least one constituent monomer of the at least one second block, and vice versa.

Each of the at least one first and/or at least one second blocks may comprise, in addition to the monomers indicated above, at least one other monomer known as at least one additional monomer, which are different from the main monomers mentioned above.

The nature and amount of the at least one additional monomer is chosen such that the block in which it is present has the desired glass transition temperature.

The at least one additional monomer may be chosen, for example, from:
a) hydrophilic monomers such as:
   ethylenically unsaturated monomers comprising at least one functional group chosen from carboxylic and sulfonic acid functional groups, such as acrylic acid, methacrylic acid, crotonic acid, maleic anhydride, itaconic acid, fumaric acid, maleic acid, acrylamidopropanesulfonic acid, vinylbenzoic acid, vinylphosphoric acid, and salts thereof;
   ethylenically unsaturated monomers comprising at least one tertiary amine functional group, such as 2-vinylpyridine, 4-vinylpyridine, dimethylaminoethyl methacrylate, diethylaminoethyl methacrylate, dimethylaminopropylmethacrylamide, and salts thereof;
methacrylates of formula CH₂═C(CH₃)—COOR₆ wherein R₆ is chosen from linear and branched alkyl groups comprising from 1 to 4 carbon atoms, such as a methyl, ethyl, propyl, and isobutyl group, wherein the alkyl groups are substituted with at least one substituent chosen from hydroxyl groups (such as 2-hydroxypropyl methacrylate and 2-hydroxyethyl methacrylate) and halogen atoms (Cl, Br, I, and F), such as trifluoroethyl methacrylate;
   methacrylates of formula CH₂═C(CH₃)—COOR₉, and
   wherein R₉ is chosen from linear and branched C₆ to C₁₂ alkyl groups, wherein at least one hetero atom chosen from O, N and S is optionally intercalated, and wherein the alkyl groups are substituted with at least one substituent chosen from hydroxyl groups and halogen atoms (Cl, Br, I, and F);
   acrylates of formula CH₂═CHCOOR₁₀,
   wherein R₁₀ is chosen from linear and branched C₁ to C₁₂ alkyl groups substituted with at least one substituent chosen from hydroxyl groups and halogen atoms (Cl, Br, I, and F), such as 2-hydroxypropyl acrylate and 2-hydroxyethyl acrylate, or R₁₀ is chosen from C₁ to C₁₂ alkyl-O—POE (polyoxyethylene) with repetition of the oxyethylene unit 5 to 30 times, for example methoxy-POE, or R₁₀ is chosen from polyoxyethylenated groups comprising from 5 to 30 ethylene oxide units; and
b) ethylenically unsaturated monomers comprising at least one silicon atom, such as methacryloxypropyltrimethoxysilane and methacryloxypropyltris(trimethylsiloxy)silane.

In some embodiments, the at least one additional monomer may be chosen from acrylic acid, methacrylic acid, and trifluoroethyl methacrylate.

According to one embodiment, the at least one pseudo-block polymer used in the at least one styling composition disclosed herein is a non-silicone polymer, i.e. a polymer free of silicon atoms.

The at least one additional monomer may be present in an amount less than or equal to 30% by weight, for example, from 1% to 30% by weight, further, for example, from 5% to 20% by weight and, even further, for example, from 7% to 15% by weight, relative to the total weight of the at least one first and/or at least one second blocks.

In some embodiments, each of the at least one first and at least one second blocks comprise at least one monomer chosen from (meth)acrylic acid esters and optionally at least one monomer chosen from (meth)acrylic acid.

For example, each of the at least one first and at least one second blocks may be totally derived from at least one monomer chosen from acrylic acid, (meth)acrylic acid esters and optionally from at least one monomer chosen from (meth)acrylic acid.

According to one embodiment, the at least one pseudo-block polymer used in the compositions disclosed herein is free of styrene. The term “polymer free of styrene” means a polymer comprising less than 10%, for example, less than 5%, further, for example, less than 2% and, even further, for example, less than 1% by weight, relative to the total weight of the polymer, of, or even comprises no, styrene monomeric units such as monomeric units of styrene and styrene derivatives, for instance methylstyrene, chlorostyrene, and chloromethylstyrene.

The at least one pseudo-block polymer used in the at least one styling composition disclosed herein may be obtained by free-radical solution polymerization according to the following preparation process:
a portion of the polymerization solvent is introduced into a suitable reactor and heated until the adequate temperature for the polymerization is reached (typically ranging from 60 to 120° C.),
once this temperature is reached, the at least one constituent monomer of the at least one first block is introduced in the presence of some of the polymerization initiator,
after a time T corresponding to a maximum degree of conversion of 90%, the at least one constituent monomer of the at least one second block and the rest of the initiator are introduced,
the mixture is left to react for a time T′ (ranging from 3 to 6 hours), after which the mixture is cooled to room temperature, and
the at least one pseudo-block polymer dissolved in the polymerization solvent is obtained.

As used herein, the term “polymerization solvent” means a solvent or a mixture of solvents. The polymerization solvent may be chosen, for example, from at least one of ethyl acetate, butyl acetate, alcohols such as isopropanol and ethanol, and aliphatic alkanes, such as isododecane. According to one embodiment, the polymerization solvent is a mixture of butyl acetate and isopropanol or isododecane.

According to one embodiment, the at least one pseudo-block polymer used in the compositions disclosed herein comprises at least one (such as one) first block with a Tg of greater than or equal to 40° C., as described above in a) and at least one (such as one) second block with a Tg of less than or equal to 20° C., as described above in b).

For example, the at least one first block with a Tg of greater than or equal to 40° C. is a copolymer derived from at least one monomer which is such that the homopolymer prepared from the at least one monomer has a glass transition temperature of greater than or equal to 40° C., such as the monomers described above.

For example, the second block with a Tg of less than or equal to 20° C. is a homopolymer derived from at least one monomer which is such that the homopolymer prepared from the at least one monomer has a glass transition temperature of less than or equal to 20° C., such as the monomers described above.

For example, the proportion of the block with a Tg of greater than or equal to 40° C. ranges from 20% to 90%, further, for example, from 30% to 80% and even, further, for example, from 50% to 70% by weight of the polymer.

For example, the proportion of the block with a Tg of less than or equal to 20° C. ranges from 5% to 75%, for example, from 15% to 50% and, further, for example, from 25% to 45% by weight of the polymer.

Thus, according to a first variant, the at least one pseudo-block polymer used in the at least one styling composition disclosed herein may comprise:
at least one first block with a Tg of greater than or equal to 40° C., for example, having a Tg ranging from 70 to 110° C., which is a methyl methacrylate/acrylic acid copolymer,
at least one second block with a Tg of less than or equal to 20° C., for example, ranging from 0 to 20° C., which is a methyl acrylate homopolymer, and
an intermediate segment that is a methyl methacrylate/acrylic acid/methyl acrylate copolymer.

According to a second variant, the at least one pseudo-block polymer used in the at least one styling composition disclosed herein may comprise:
at least one first block with a Tg of greater than or equal to 40° C., for example, ranging from 70 to 100° C., which is a methyl methacrylate/acrylic acid/trifluoroethyl methacrylate copolymer,
at least one second block with a Tg of less than or equal to 20° C., for example, ranging from 0 to 20° C., which is a methyl acrylate homopolymer, and
an intermediate segment that is a methyl methacrylate/acrylic acid/methyl acrylate/trifluoroethyl methacrylate random copolymer.

According to a third variant, the at least one pseudo-block polymer used in the at least one styling composition disclosed herein may comprise:
at least one first block with a Tg of greater than or equal to 40° C., for example, ranging from 85 to 115° C., which is an isobornyl acrylate/isobutyl methacrylate copolymer,
at least one second block with a Tg of less than or equal to 20° C., for example, ranging from −85 to −55° C., which is a 2-ethylhexyl acrylate homopolymer, and
an intermediate segment, which is an isobornyl acrylate/isobutyl methacrylate/2-ethylhexyl acrylate random copolymer.

According to a fourth variant, the at least one pseudo-block polymer used in the at least one styling composition disclosed herein may comprise:
at least one first block with a Tg of greater than or equal to 40° C., for example, ranging from 85 to 115° C., which is an isobornyl acrylate/methyl methacrylate copolymer,
at least one second block with a Tg of less than or equal to 20° C., for example, ranging from −85 to −55° C., which is a 2-ethylhexyl acrylate homopolymer, and
an intermediate segment that is an isobornyl acrylate/methyl methacrylate/2-ethylhexyl acrylate random copolymer.

According to a fifth variant, the at least one pseudo-block polymer used in the at least one styling composition disclosed herein may comprise:
at least one first block with a Tg of greater than or equal to 40° C., for example, ranging from 95 to 125° C., which is an isobornyl acrylate/isobornyl methacrylate copolymer,
at least one second block with a Tg of less than or equal to 20° C., for example, ranging from −85 to −55° C., which is a 2-ethylhexyl acrylate homopolymer, and
an intermediate segment that is an isobornyl acrylate/isobornyl methacrylate/2-ethylhexyl acrylate random copolymer.

According to a sixth variant, the at least one pseudo-block polymer used in the at least one styling composition disclosed herein may comprise:
at least one first block with a Tg of greater than or equal to 40° C., for example, ranging from 85 to 115° C., which is an isobornyl methacrylate/isobutyl methacrylate copolymer,
at least one second block with a Tg of less than or equal to 20° C., for example, ranging from −35 to −5° C., which is an isobutyl acrylate homopolymer, and
an intermediate segment that is an isobornyl methacrylate/isobutyl methacrylate/isobutyl acrylate random copolymer.

According to a seventh variant, the at least one pseudo-block polymer used in the at least one styling composition disclosed herein may comprise:
at least one first block with a Tg of greater than or equal to 40° C., for example, ranging from 95 to 125° C., which is an isobornyl acrylate/isobornyl methacrylate copolymer,
at least one second block with a Tg of less than or equal to 20° C., for example, ranging from −35 to −5° C., which is an isobutyl acrylate homopolymer, and
an intermediate segment that is an isobornyl acrylate/isobornyl methacrylate/isobutyl acrylate random copolymer.

According to an eighth variant, the at least one pseudo-block polymer used in the at least one styling composition disclosed herein may comprise:
at least one first block with a Tg of greater than or equal to 40° C., for example, ranging from 60 to 90° C., which is an isobornyl acrylate/isobutyl methacrylate copolymer,
at least one second block with a Tg of less than or equal to 20° C., for example, ranging from −35 to −5° C., which is an isobutyl acrylate homopolymer, and
an intermediate segment that is an isobornyl acrylate/isobutyl methacrylate/isobutyl acrylate random copolymer.

According to a ninth variant, the at least one pseudo-block polymer used in the at least one styling composition disclosed herein may comprise:
at least one first block with a Tg of greater than or equal to 40° C., for example, with a Tg ranging from 70 to 110° C., which is an acrylic acid/methyl acrylate copolymer,
at least one second block with a Tg of less than or equal to 20° C., for example, ranging from 0 to 20° C., which is a methyl acrylate homopolymer, and
an intermediate segment that is an acrylic acid/methyl acrylate/polymethyl acrylate copolymer.

According to a another embodiment, the at least one pseudo-block polymer used in the compositions disclosed herein comprises at least one (such as one) first block with a glass transition temperature (Tg) ranging from 20 to 40° C., in accordance with the blocks described in c) and at least one (such as one) second block with a glass transition temperature of less than or equal to 20° C., as described above in b) or a glass transition temperature of greater than or equal to 40° C., as described in a) above.

For example, the proportion of the at least one first block with a Tg ranging from 20 to 40° C. ranges from 10% to 85%, further, for example, from 30% to 80% and even further, for example, from 50% to 70% by weight of the polymer.

When the at least one second block is a block with a Tg of greater than or equal to 40° C., it may, for example, be present in a proportion ranging from 10% to 85%, further, for example, from 20% to 70% and, even further, for example, from 30% to 70% by weight of the polymer.

When the at least one second block is a block with a Tg of less than or equal to 20° C., it may, for example, be present in a proportion ranging from 10% to 85%, better still from 20% to 70% and even better still from 20% to 50% by weight of the polymer.

For example, the at least one first block with a Tg ranging from 20 to 40° C. is a copolymer derived from at least one monomer which is such that the corresponding homopolymer has a Tg of greater than or equal to 40° C., and from at least one monomer which is such that the corresponding homopolymer has a Tg of less than or equal to 20° C.

For example, the at least one second block with a Tg of less than or equal to 20° C. or with a Tg of greater than or equal to 40° C. may be a homopolymer.

Thus, according to a first variant of this embodiment, the at least one pseudo-block polymer used in the at least one styling composition disclosed herein may comprise:
at least one first block with a Tg ranging from 20 to 40° C., for example, with a Tg of 25 to 39° C., which is a copolymer comprising at least one methyl acrylate monomer, at least one methyl methacrylate monomer and at least one acrylic acid monomer,
at least one second block with a Tg of greater than or equal to 40° C., for example, ranging from 85 to 125° C., which is a homopolymer comprising methyl methacrylate monomers, and
an intermediate segment comprising at least one methyl acrylate or methyl methacrylate monomer, and
an intermediate segment comprising methyl methacrylate, at least one acrylic acid monomer and at least one methyl acrylate monomer.

According to a second variant of this embodiment, the at least one pseudo-block polymer used in the at least one styling composition disclosed herein may comprise:
at least one first block with a Tg ranging from 20 to 40° C., for example, with a Tg ranging from 21 to 39° C., which is a copolymer comprising isobornyl acrylate/isobutyl methacrylate/2-ethylhexyl acrylate,
at least one second block with a Tg of less than or equal to 20° C., for example, ranging from −65 to −35° C., which is a methyl methacrylate homopolymer, and
an intermediate segment that is an isobornyl acrylate/isobutyl methacrylate/2-ethylhexyl acrylate random copolymer.

According to a third variant of this embodiment, the at least one pseudo-block polymer used in the at least one styling composition disclosed herein may comprise:
at least one first block with a Tg ranging from 20 to 40° C., for example, with a Tg ranging from 21 to 39° C., which is an isobornyl acrylate/methyl acrylate/acrylic acid copolymer,
at least one second block with a Tg of greater than or equal to 40° C., for example, ranging from 85 to 115° C., which is an isobornyl acrylate homopolymer, and
an intermediate segment that is an isobornyl acrylate/methyl acrylate/acrylic acid random copolymer.

The at least one styling composition disclosed herein, for example, comprises the at least one pseudo-block polymer in an amount ranging from 0.1% to 60% by weight, further, for example, from 0.5% to 50% by weight and, even further, for example, from 1% to 40% by weight, relative to the total weight of the at least one styling composition.

Additional Fixing Polymer

The at least one styling composition for the aerosol device also comprises a second fixing polymer that is different from the pseudo-block polymer used; this second fixing polymer is referred to in the description hereinbelow as the at least one additional fixing polymer.

The at least one additional fixing polymer may be chosen from anionic, cationic, amphoteric, and nonionic fixing polymers used in the art.

The at least one additional fixing polymer may be soluble in the cosmetically acceptable medium or insoluble in this same medium and, in this case, used in the form of dispersions of solid or liquid polymer particles (latices or pseudolatices).

The anionic fixing polymers generally used are polymers comprising groups derived from carboxylic acid, sulfonic acid or phosphoric acid and have a number-average molecular mass ranging from 500 to 5,000,000.

The carboxylic groups are provided by unsaturated monocarboxylic or dicarboxylic acid monomers such as those corresponding to the formula: 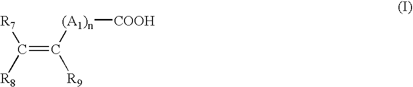
wherein: n is an integer ranging from 0 to 10, A₁ is a methylene group, optionally connected to the carbon atom of the unsaturated group, or to the neighbouring methylene group when n is greater than 1, via a hetero atom such as oxygen or sulphur; R₇ is chosen from a hydrogen atom and phenyl and benzyl groups; R₈ is chosen from a hydrogen atom and lower alkyl and carboxyl groups; and R₉ is chosen from a hydrogen atom, lower alkyl groups and —CH₂—COOH, phenyl and benzyl groups.

In the abovementioned formula, a lower alkyl group may, for example, be chosen from alkyl groups comprising 1 to 4 carbon atoms, such as methyl and ethyl groups.

Examples of the anionic fixing polymers comprising carboxylic groups that may be used include:
A) acrylic or methacrylic acid homo- or copolymers, or salts thereof, for example, the products sold under the names VERSICOL® E or K by the company Allied Colloid and ULTRAHOLD® by the company BASF, the copolymers of acrylic acid and of acrylamide sold in the form of their sodium salts under the names RETEN 421, 423 or 425 by the company Hercules, the sodium salts of polyhydroxycarboxylic acids.
B) copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described, for example, in French Patent No. 1 222 944 and German Patent Application No. 2 330 956, the copolymers of this type comprising an optionally N-alkylated and/or hydroxyalkylated acrylamide unit in their chain as described, for example, in Luxembourg Patent Application Nos. 75370 and 75371 or sold under the name QUADRAMER by the company American Cyanamid. Mention may also be made of copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by the company ISP under the name ACRYLIDONE® LM and methacrylic acid/ethyl acrylate/tert-butyl acrylate terpolymers, such as the product sold under the name LUVIMER® 100 P by the company BASF.

Mention may also be made of methacrylic acid/acrylic acid/ethyl acrylate/methyl methacrylate copolymers as an aqueous dispersion, sold under the name AMERHOLD® DR 25 by the company Amerchol.
C) crotonic acid copolymers, such as those comprising vinyl acetate or propionate units in their chain and optionally other monomers such as allylic esters or methallylic esters, vinyl ether or vinyl ester of a linear or branched saturated carboxylic acid with a long hydrocarbon chain such as those comprising at least 5 carbon atoms, it being possible for these polymers optionally to be grafted or crosslinked, or alternatively another vinyl, allylic or methallylic ester monomer of an α- or β-cyclic carboxylic acid. Such polymers are described, inter alia, in French Patent Nos. 1 222 944, 1 580 545, 2 265 782, 2 265 781, 1 564 110, and 2 439 798. Commercial products falling into this class are the resins 28-29-30, 26-13-14 and 28-13-10 sold by the company National Starch.
D) copolymers derived from C₄-C₈ monounsaturated carboxylic acids or anhydrides chosen from:
   copolymers comprising (i) at least one monomeric unit chosen from maleic, fumaric and itaconic acids and anhydrides and (ii) at least one monomeric unit chosen from vinyl esters, vinyl ethers, vinyl halides, phenylvinyl derivatives, acrylic acid and esters thereof, wherein the anhydride functional groups of these copolymers are optionally monoesterified or monoamidated. Such polymers are described, for example, in U.S. Pat. Nos. 2,047,398, 2,723,248 and 2,102,113 and GB Patent No. 839 805. Commercial products are, for example, those sold under the names GANTREZ® AN or ES by the company ISP,
   copolymers comprising (i) at least one monomeric unit chosen from maleic, citraconic and itaconic anhydride units and (ii) at least one monomeric unit chosen from allylic and methallylic esters optionally comprising at least one group chosen from acrylamide, methacrylamide, α-olefin, acrylic, and methacrylic esters, acrylic and methacrylic acid and vinylpyrrolidone groups in their chain, wherein the anhydride functional groups of these copolymers are optionally monoesterified or monoamidated.

These polymers are described, for example, in French Patent Nos. 2 350 384 and 2 357 241.
E) Polyacrylamides comprising carboxylate groups.

The homopolymers and copolymers comprising sulfonic groups are polymers comprising at least one monomeric unit chosen from vinylsulfonic, styrenesulfonic, naphthalenesulfonic, and acrylamidoalkylsulfonic units.

These polymers can be chosen, for example, from:
polyvinylsulfonic acid salts having a molecular mass ranging from 1,000 to 100,000, as well as the copolymers with an unsaturated comonomer such as acrylic and methacrylic acids and esters thereof, as well as acrylamide and derivatives thereof, and vinyl ethers and vinylpyrrolidone;
polystyrenesulfonic acid salts such as the sodium salts that are sold for example under the names FLEXAN® 500 and FLEXAN® 130 by National Starch. These compounds are described in Patent No. FR 2 198 719;
polyacrylamidesulfonic acid salts, such as those mentioned in U.S. Pat. No. 4,128,631 and, for example, polyacrylamidoethylpropanesulfonic acid sold under the name COSMEDIA POLYMER HSP 1180 by Henkel.

As another anionic fixing polymer that can be used in the at least one styling composition disclosed herein mention may be made of the branched block anionic polymer sold under the name FIXATE G-100 by the company Noveon.

In some embodiments, the anionic fixing polymers may be, for example, chosen from acrylic acid copolymers, such as the acrylic acid/ethyl acrylate/N-tert-butylacrylamide terpolymers sold, for example, under the name ULTRAHOLD® STRONG by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold, for example, under the name Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the name GANTREZ® ES 425 by the company ISP, the copolymers of methacrylic acid and of methyl methacrylate sold under the name EUDRAGIT® L by the company Rohm Pharma, the copolymers of methacrylic acid and of ethyl acrylate sold under the name LUVIMER® MAEX or MAE by the company BASF, the vinyl acetate/crotonic acid copolymers sold under the name LUVISET CA 66 by the company BASF, the vinyl acetate/crotonic acid copolymers grafted with polyethylene glycol sold under the name ARISTOFLEX® A by the company BASF, and the polymer sold under the name FIXATE G-100 by the company Noveon.

According to one embodiment, the anionic fixing polymers are chosen from methyl vinyl ether/monoesterified maleic anhydride copolymers sold under the name GANTREZ® ES 425 by the company ISP, the acrylic acid/ethyl acrylate/N-tert-butylacrylamide terpolymers sold under the name ULTRAHOLD® STRONG by the company BASF, the copolymers of methacrylic acid and of methyl methacrylate sold under the name EUDRAGIT® L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name LUVIMER® MAEX or MAE by the company BASF, the vinylpyrrolidone/acrylic acid/lauryl methacrylate terpolymers sold under the name ACRYLIDONE® LM by the company ISP, and the polymer sold under the name FIXATE G-100 by the company Noveon.

The cationic fixing polymers that may be used in the at least one styling composition disclosed herein may be chosen from polymers comprising primary, secondary, tertiary and/or quaternary amine groups forming part of the polymer chain or directly attached thereto, and having a molecular weight ranging from 500 to 5,000,000 and, for example, from 1,000 to 3,000,000.

Among these polymers, mention may be made, for example, of the following cationic polymers:
(1) homopolymers or copolymers derived from acrylic or methacrylic esters or amides and comprising at least one of the units of the following formulae: 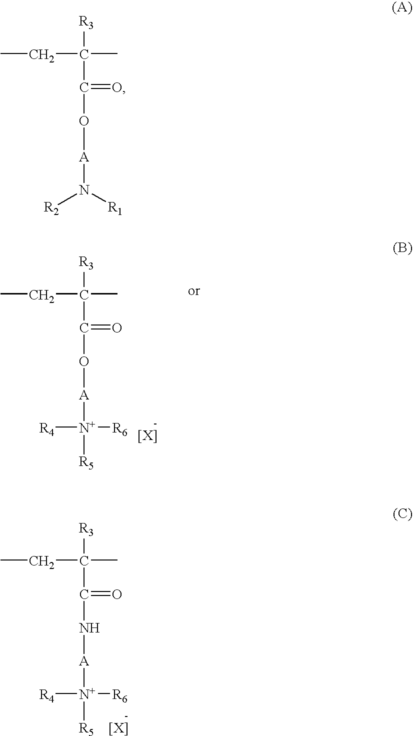
wherein:
   R₃, which may be identical or different, is chosen from a hydrogen atom and a CH₃ radical;
   A, which may be identical or different, is chosen from linear and branched alkyl groups comprising 1 to 6 carbon atoms and hydroxyalkyl groups comprising 1 to 4 carbon atoms;
   R₄, R₅ and R₆, which may be identical or different, are each chosen from alkyl groups comprising from 1 to 18 carbon atoms and benzyl radicals;
   R₁ and R₂, which may be identical or different, are each chosen from hydrogen atoms and alkyl groups comprising from 1 to 6 carbon atoms;
   X, which may be identical or different, is chosen from a methosulfate anion and halides such as chloride or bromide.

The copolymers of the family (1) also comprise at least one comonomer unit that may be chosen from acrylamides, methacrylamides, diacetone acrylamides, acrylamides and methacrylamides substituted on the nitrogen with at least one substituent chosen from lower (C₁₋₄) alkyl groups, groups derived from acrylic or methacrylic acids or esters thereof, vinyllactams such as vinylpyrrolidone and vinylcaprolactam, and vinyl esters.

Thus, among these copolymers of the family (1), mention may be made of:
copolymers of acrylamide and of dimethylaminoethyl methacrylate quaternized with dimethyl sulfate or with a dimethyl halide, such as the product sold under the name HERCOFLOC® by the company Hercules,
copolymers of acrylamide and of methacryloyloxyethyltrimethylammonium chloride, described, for example, in Patent Application No. EP-A-080 976 and sold under the name BINA QUAT P 100 by the company Ciba Geigy,
copolymers of acrylamide and of methacryloyloxyethyltrimethylammonium methosulfate, such as the product sold under the name RETEN by the company Hercules,
quaternized or non-quaternized vinylpyrrolidone/dialkylaminoalkyl acrylate or methacrylate copolymers, such as the products sold under the name “GAFQUAT®” by the company ISP, such as, for example, “GAFQUAT® 734” or “GAFQUAT® 755”, or alternatively the products known as “COPOLYMER® 845, 958 and 937”. These polymers are described in detail in French Patent Nos. 2 077 143 and 2 393 573,
fatty-chain polymers comprising at least one vinylpyrrolidone unit, such as the products sold under the name STYLEZE W20 and STYLEZE W10 by the company ISP,
dimethylaminoethyl methacrylate/vinylcaprolactam/vinylpyrrolidone terpolymers, such as the product sold under the name GAFFIX VC 713 by the company ISP, and
quaternized vinylpyrrolidone/dimethylaminopropylmethacrylamide copolymers, such as the products sold under the name “GAFQUAT® HS 100” by the company ISP;
(2) cationic polysaccharides, for example, comprising quaternary ammonium, such as those described in U.S. Pat. Nos. 3,589,578 and 4,031,307, such as guar gums comprising trialkylammonium cationic groups. Such products are sold, for example, under the trade names JAGUAR C13 S, JAGUAR C 15 and JAGUAR C 17 by the company Meyhall;
(3) quaternary copolymers of vinylpyrrolidone and of vinylimidazole;
(4) chitosans or salts thereof; the salts that can be used may, for example, be chosen from chitosan acetate, lactate, glutamate, gluconate, and pyrrolidonecarboxylate.

Among these compounds, mention may be made of chitosan having a degree of deacetylation of 90.5% by weight, sold under the name KYTAN BRUT STANDARD by the company Aber Technologies, and chitosan pyrrolidonecarboxylate sold under the name KYTAMER® PC by the company Amerchol.
(5) cationic cellulose derivatives such as copolymers of cellulose or of cellulose derivatives grafted with a water-soluble monomer comprising a quaternary ammonium, and disclosed, for example, in U.S. Pat. No. 4,131,576, such as hydroxyalkylcelluloses, for instance hydroxymethyl-, hydroxyethyl- or hydroxypropylcelluloses grafted, for example, with a methacryloyloxyethyltrimethylammonium, methacrylamidopropyltrimethylammonium or dimethyldiallylammonium salt.

The products sold corresponding to this definition are, for example, the products sold under the name “CELQUAT L 200” and “CELQUAT H 100” by the company National Starch.

The amphoteric fixing polymers that can be used in the at least one styling composition disclosed herein may be chosen from polymers comprising units B and C distributed randomly in the polymer chain, wherein B is chosen from units derived from a monomer comprising at least one basic nitrogen atom and C is chosen from units derived from an acid monomer comprising at least one group chosen from carboxylic and sulfonic groups, or alternatively B and C may be chosen from groups derived from carboxybetaine and sulfobetaine zwitterionic monomers;
B and C can also be chosen from cationic polymer chains comprising at least one group chosen from primary, secondary, tertiary and quaternary amine groups, wherein at least one of the amine groups bears a carboxylic or sulfonic group connected via a hydrocarbon group or alternatively B and C form part of a chain of a polymer comprising an α,β-dicarboxylic ethylene unit in which one of the carboxylic groups has been made to react with a polyamine comprising at least one group chosen from primary and secondary amine groups.

According to one embodiment, the amphoteric fixing polymers corresponding to the definition given above may be chosen from the following polymers:
(1) copolymers having acidic vinyl and basic vinyl units, such as those resulting from the copolymerization of a monomer derived from a vinyl compound bearing a carboxylic group such as acrylic acid, methacrylic acid, maleic acid, α-chloroacrylic acid, and a basic monomer derived from a substituted vinyl compound comprising at least one basic atom, such as dialkylaminoalkyl methacrylate and acrylate, dialkylaminoalkylmethacrylamides and -acrylamides. Such compounds are described in U.S. Pat. No. 3,836,537.
(2) polymers comprising units derived from:
   a) at least one monomer chosen from acrylamides and methacrylamides substituted on the nitrogen atom with an alkyl group,
   b) at least one acidic comonomer comprising at least one reactive carboxylic group, and
   c) at least one basic comonomer, such as esters comprising at least one substituent chosen from primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides that may be used in the styling composition disclosed herein may be chosen from compounds wherein the alkyl groups comprise from 2 to 12 carbon atoms and, for example, N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen, for example, from acrylic acid, methacrylic acid, crotonic acid, itaconic acid, maleic acid and fumaric acid and alkyl monoesters, comprising from 1 to 4 carbon atoms, of maleic and fumaric acids and anhydrides.

For example, the basic comonomers may be chosen from aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold under the name AMPHOMER® or LOVOCRYL® 47 by the company National Starch, may, for example, be used.
(3) crosslinked and acylated polyamino amides partially or totally derived from polyamino amides of general formula: [symbol]
   CO—R₁₀—CO-Z [symbol] (II)
   wherein R₁₀ is chosen from divalent groups derived from saturated dicarboxylic acids, mono- or dicarboxylic aliphatic acids comprising an ethylenic double bond, esters of a lower alkanol, comprising from 1 to 6 carbon atoms, of these acids, or groups derived from the addition of any one of the acids to a bis primary) or bis(secondary) amine, and Z is chosen from groups derived from a bis(primary), mono- and bis(secondary) polyalkylene-polyamine and, for example, represents:
a) in proportions ranging from 60 to 100 mol %, the group: 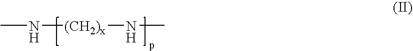
wherein x=2 and p=2 or 3, or alternatively x=3 and p=2
wherein the group is derived from a compound chosen from diethylenetriamine, triethylenetetraamine, and dipropylenetriamine;
b) in proportions ranging from 0 to 40 mol %, the group (II) above in which x=2 and p=1 and which is derived from a compound chosen from ethylenediamine and piperazine: 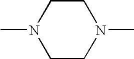
c) in proportions ranging from 0 to 20 mol %, the —NH(CH₂)₆—NH— group, which is derived from hexamethylenediamine, these polyamino amides can be crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyamino amide and acylated by the action of acrylic acid, chloroacetic acid or an alkane sultone, or salts thereof.

The saturated carboxylic acids may, for example, be chosen from acids comprising 6 to 10 carbon atoms, such as adipic acid, 2,2,4-trimethyladipic acid and 2,4,4-trimethyladipic acid, terephthalic acid, acids comprising an ethylenic double bond such as acrylic acid, methacrylic acid and itaconic acid.

The alkane sultones used in the acylation may, for example, be chosen from propane sultone or butane sultone; the salts of the acylating agents may, for example, be chosen from sodium and potassium salts.
(4) polymers comprising zwitterionic units of formula: 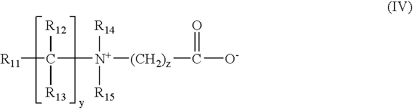
   wherein R₁₁ is chosen from polymerizable unsaturated groups, such as acrylate, methacrylate, acrylamide and methacrylamide groups, y and z are each chosen from integers ranging from 1 to 3, R₁₂ and R₁₃, which may be identical or different, are each chosen from hydrogen atoms and methyl, ethyl and propyl groups, and R₁₄ and R₁₅, which may be identical or different, are each chosen from hydrogen atoms and alkyl groups such that the sum of the carbon atoms in R₁₄ and R₁₅ does not exceed 10.

The polymers comprising such units can also comprise units derived from non-zwitterionic monomers, such as monomers chosen from dimethyl- and diethylaminoethyl acrylate and methacrylate, alkyl acrylates and methacrylates, acrylamides and methacrylamides, and vinyl acetate.

By way of example, mention may be made of the copolymers of methyl methacrylate/methyl dimethylcarboxymethylammonioethyl methacrylate such as the product sold under the name DIAFORMER Z301 by the company Sandoz.
(5) polymers derived from chitosan comprising monomer units corresponding to the following formulae: 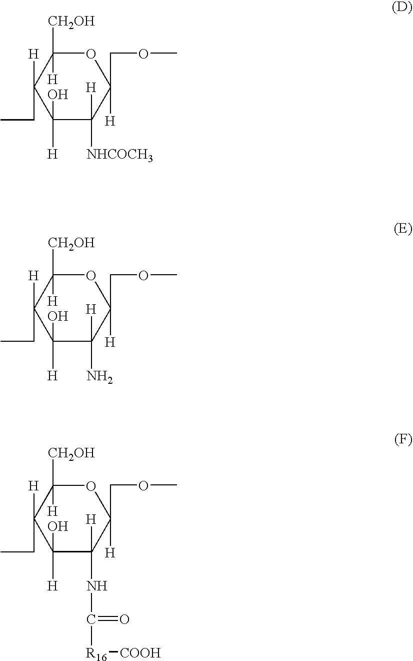
   wherein the unit (D) is present in proportions ranging from 0 to 30%, the unit (E) in proportions ranging from 5 to 50% and the unit (F) in proportions ranging from 30% to 90%, it being understood that, in unit (F), R₁₆ is a group of formula: 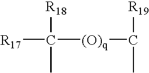
   wherein, if q=0, R₁₇, R₁₈ and R₁₉, which may be identical or different, are each chosen from hydrogen atoms, methyl, hydroxyl, acetoxy and amino residues, and monoalkylamine and dialkylamine residues that are optionally interrupted by at least one nitrogen atom and/or optionally substituted with at least one group chosen from amine, hydroxyl, carboxyl, alkylthio, sulfonic groups, and alkylthio residues wherein the alkyl group bears an amino residue, at least one of the groups R₁₇, R₁₈ and R₁₉ being, in this case, a hydrogen atom;
or, if q=1, R₁₇, R₁₈ and R₁₉ are each a hydrogen atom, as well as the salts formed by these compounds with bases or acids.
(6) polymers corresponding to the general formula (V) that are described, for example, in French Patent No. 1 400 366: 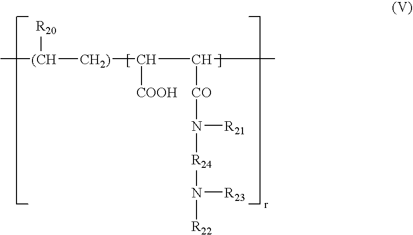
   wherein R₂₀ is chosen from a hydrogen atom, CH₃O, CH₃CH₂O, and phenyl groups, R₂₁ is chosen from a hydrogen atom and lower alkyl groups, such as methyl and ethyl, R₂₂ is chosen from a hydrogen atom and C₁₆ lower alkyl groups, such as methyl and ethyl, R₂₃ is chosen from C₁₋₆ lower alkyl groups, such as methyl, ethyl, and groups corresponding to the formula: —R₂₄—N(R₂₂)₂, wherein R₂₄ is chosen from —CH₂—CH₂—, —CH₂—CH₂—CH₂— and —CH₂—CH(CH₃)— group, and R₂₂ is chosen from a hydrogen atom and C₁₋₆ lower alkyl groups, such as methyl and ethyl.
(7) polymers derived from the N-carboxyalkylation of chitosan, such as N-carboxymethylchitosan or N-carboxybutylchitosan sold under the name “EVALSAN” by the company Jan Dekker.
(8) amphoteric polymers of the type -D-X-D-X— chosen from:
   a) polymers obtained by the action of chloroacetic acid or sodium chloroacetate on compounds comprising at least one unit of formula:
      -D-X-D-X-D- (VI)
      wherein D is a group 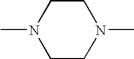
      and X is chosen from symbols E and E′, wherein E or E′, which may be identical or different, are divalent groups chosen from alkylene groups comprising a straight or branched chain comprising up to 7 carbon atoms in the main chain, wherein the divalent groups are optionally substituted with at least one hydroxyl group. E or E′ can additionally comprise at least one atom chosen from oxygen, nitrogen and sulfur atoms and 1 to 3 rings chosen from aromatic and heterocyclic rings; wherein the oxygen, nitrogen and sulfur atoms are present in the form of at least one group chosen from ether, thioether, sulfoxide, sulfone, sulfonium, alkylamine and alkenylamine groups, hydroxyl, benzylamine, amine oxide, quaternary ammonium, amide, imide, alcohol, ester and urethane groups;
b) polymers of formula:
   -D-X-D-X— (VI′)
   wherein D is a group 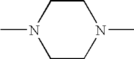
   and X is chosen from symbols E or E′ and at least one X is chosen from E′; E having the meaning given above and E′ is a divalent alkylene group comprising a straight or branched chain comprising up to 7 carbon atoms in the main chain, wherein the divalent alkylene group is optionally substituted with at least one hydroxyl group. E′ can also comprise at least one nitrogen atom, wherein the nitrogen atom is substituted with an alkyl chain that is optionally interrupted by an oxygen atom and comprises at least one functional group chosen from carboxyl functional groups and hydroxyl functional groups, and wherein the alkyl chain is betainized by reaction with chloroacetic acid or sodium chloroacetate.
(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also comprise other vinyl comonomers, such as vinylcaprolactam.

According to one embodiment, the amphoteric fixing polymers are chosen from those of family (3), such as the copolymers whose CTFA name is octylacryl-amide/acrylates/butylaminoethyl methacrylate copolymer, such as the products sold under the names AMPHOMER®, AMPHOMER® LV 71 or LOVOCRYL® 47 by the company National Starch and those of family (4) such as the copolymers of methyl methacrylate/methyl dimethylcarboxymethylammonioethyl methacrylate, sold, for example, under the name DIAFORMER Z301 by the company Sandoz.

The nonionic fixing polymers that may be used in the styling composition disclosed herein may be chosen, for example, from:
polyalkyloxazolines;
vinyl acetate homopolymers;
vinyl acetate copolymers, for instance copolymers of vinyl acetate and of acrylic ester; copolymers of vinyl acetate and of ethylene, and copolymers of vinyl acetate and of maleic ester, for example, of dibutyl maleate;
homopolymers and copolymers of acrylic esters, for instance copolymers of alkyl acrylates and of alkyl methacrylates, such as the products sold by the company Rohm & Haas under the names PRIMAL® AC-261 K and EUDRAGIT® NE 30 D, by the company BASF under the name 8845, or by the company Hoechst under the name APPRETAN® N9212;
copolymers of acrylonitrile and of a nonionic monomer chosen, for example, from butadiene and alkyl(meth)acrylates; mention may be made of the products sold under the name CJ 0601 B by the company Rohm & Haas;
styrene homopolymers;
styrene copolymers, for instance copolymers of styrene and of an alkyl (meth)acrylate, such as the products MOWILITH® LDM 6911, MOWILITH® DM 611 and MOWILITH® LDM 6070 sold by the company Hoechst, and the products RHODOPAS® SD 215 and RHODOPAS® DS 910 sold by the company Rhône-Poulenc; copolymers of styrene, of alkyl methacrylate and of alkyl acrylate; copolymers of styrene and of butadiene; and copolymers of styrene, of butadiene and of vinylpyridine;
polyamides;
vinyllactam homopolymers other than vinylpyrrolidone homopolymers, such as the polyvinylcaprolactam sold under the name LUVISKOL® Plus by the company BASF; and
vinyllactam copolymers such as a poly(vinylpyrrolidone/vinyllactam) copolymer sold under the trade name LUVITEC® VPC 55K65W by the company BASF, poly(vinylpyrrolidone/vinyl acetate) copolymers, such as those sold under the name PVPVA® S630L by the company ISP, LUVISKOL® VA 73, VA 64, VA 55, VA 37 and VA 28 by the company BASF; and poly(vinylpyrrolidone/vinyl acetate/vinyl propionate) terpolymers, for instance the product sold under the name LUVISKOL® VAP 343 by the company BASF.

The alkyl groups of the nonionic polymers mentioned above may, for example, comprise from 1 to 6 carbon atoms.

According to one embodiment, it is also possible to use grafted silicone fixing polymers comprising a polysiloxane portion and a portion comprising a non-silicone organic chain, one of the two portions constituting the main chain of the polymer, and the other being grafted onto the main chain.

These polymers are described, for example, in Patent Application Nos. EP-A-0 412 704, EP-A-0 412 707, EP-A-0 640 105 and WO 95/00578, EP-A-0 582 152 and WO 93/23009 and U.S. Pat. No. 4,693,935, U.S. Pat. No. 4,728,571 and U.S. Pat. No. 4,972,037.

The grafted silicone polymers may be chosen from amphoteric, anionic, and non-ionic polymers, and, in some embodiments, may be chosen from anionic and non-ionic polymers.

Such polymers are, for example, copolymers that can be obtained by free radical polymerization from the monomer mixture formed from:
a) 50 to 90% by weight of tert-butyl acrylate;
b) 0 to 40% by weight of acrylic acid; and
c) 5 to 40% by weight of a silicone macromer of formula: 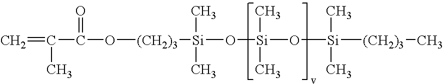
   wherein v is a number ranging from 5 to 700, the weight percentages being calculated relative to the total weight of the monomers.

Other examples of grafted silicone polymers include polydimethylsiloxanes (PDMSs) onto which are grafted, via a thiopropylene-type connecting chain, mixed polymer units of poly(meth)acrylic acid and of polyalkyl(meth)acrylate and polydimethylsiloxanes (PDMSs) onto which are grafted, via a thiopropylene connecting chain, polymer units of polyisobutyl(meth)acrylate.

Another type of silicone fixing polymer that may be mentioned is the product LUVIFLEX® SILK, sold by the company BASF.

The at least one additional fixing polymer may also be chosen from functionalized and non-functionalized, silicone and non-silicone, cationic, nonionic, anionic and amphoteric polyurethanes.

According to one embodiment, the at least one additional fixing polymer is chosen from polyurethanes disclosed in Patent Application Nos. EP 0 751 162, EP 0 637 600, EP 0 648 485 and FR 2 743 297, and Patent Application Nos. EP 0 656 021 and WO 94/03510 from the company BASF and EP 0 619 111 from the company National Starch.

As examples of polyurethanes that may be used, mention may be made of the products sold under the names LUVISET PUR® and LUVISET® SI PUR by the company BASF.

The at least one additional fixing polymer may be present in an amount ranging from 0.1% to 20% by weight and, further, for example, from 0.5% to 10% by weight, relative to the total weight of the at least one styling composition.

The at least one styling composition may also comprise at least one additive chosen from silicones soluble or insoluble, dispersed and microdispersed form, thickening polymers, gelling agents, nonionic, anionic, cationic and amphoteric surfactants, ceramides and pseudoceramides, vitamins and provitamins, including panthenol, plant, animal, mineral and synthetic oils, waxes other than ceramides and pseudoceramides, water-soluble and liposoluble, silicone and non-silicone sunscreens, glycerol, permanent and temporary dyes, nacreous agents and opacifiers, sequestering agents, plasticizers, solubilizers, pH modifiers, mineral thickeners, antioxidants, hydroxy acids, penetrating agents, fragrances, fragrance solubilizers (peptizers), preserving agents, and anticorrosion agents.

The at least one additive may be present in an amount ranging from 0 to 20% by weight, relative to the total weight of the at least one styling composition.

A person skilled in the art will take care to select the optional additives and the amount thereof such that they do not harm the properties of the at least one styling composition disclosed herein.

According to one embodiment, the at least one styling composition comprises at least one additional cosmetic additive chosen from thickening polymers, gelling agents and surfactants.

In one embodiment, the at least one styling composition disclosed herein comprises at least one thickener, also known as a “rheology modifier”.

The rheology modifiers may be chosen from fatty acid amides (coconut monoethanolamide, diethanolamide, and oxyethylenated carboxylic acid alkyl ether monoethanolamide), cellulose-based thickeners (hydroxyethylcellulose, hydroxypropylcellulose and carboxymethylcellulose), guar gum and derivatives thereof (hydroxypropyl guar), gums of microbial origin (xanthan gum and scleroglucan gum), acrylic acid and acrylamidopropanesulfonic acid crosslinked homopolymers and copolymers, and associative thickening polymers as described below.

These associative polymers are water-soluble polymers that are capable, in an aqueous medium, of reversibly associating with each other or with other molecules.

Their chemical structure comprises hydrophilic zones and hydrophobic zones characterized by at least one fatty chain.

The associative polymers may be chosen from anionic, cationic, amphoteric and nonionic polymers.

The associative thickeners may be present in an amount ranging from 0.01% to 10% by weight and, for example, 0.1% to 5% by weight, relative to the total weight of the styling composition.

Examples of anionic associative polymers include:
(I) polymers comprising at least one hydrophilic unit and at least one fatty-chain allyl ether unit, for example, polymers whose hydrophilic unit comprises at least one ethylenic unsaturated anionic monomer, for example, chosen from vinylcarboxylic acid and, further, for example, acrylic acids and methacrylic acids, wherein the fatty-chain allyl ether unit corresponds to the monomer of formula (XV) below:
   CH₂═CR′CH₂OBₙR (XV)
wherein R′ is chosen from H and CH₃, B is an ethyleneoxy radical, n is zero or is an integer ranging from 1 to 100, R is chosen from hydrocarbon-based radicals chosen from alkyl, arylalkyl, aryl, alkylaryl and cycloalkyl radicals, comprising from 8 to 30 carbon atoms, such as 10 to 24 carbon atoms and even further, such as from 12 to 18 carbon atoms. In one embodiment, a unit of formula (XV) that may be used is a unit in which R′ is H, n is equal to 10 and R is a stearyl (C₁₈) radical.

Anionic associative polymers of this type are described and prepared, according to an emulsion polymerization process, in Patent No. EP-0 216 479.

According to another embodiment, the anionic associative polymers may be chosen from polymers formed from 20% to 60% by weight of acrylic acid and/or of methacrylic acid, from 5% to 60% by weight of lower alkyl(meth)acrylates, from 2% to 50% by weight of fatty-chain allyl ether of formula (XV), and from 0% to 1% by weight of a crosslinking agent which is a well-known copolymerizable unsaturated polyethylenic monomer, such as diallyl phthalate, allyl(meth)acrylate, divinylbenzene, (poly)ethylene glycol dimethacrylate and methylenebisacrylamide.

Among the latter polymers, those that may, for example, be mentioned include crosslinked terpolymers of methacrylic acid, of ethyl acrylate and of polyethylene glycol (10 EO) stearyl alcohol ether (Steareth-10), such as those sold by the company Allied Colloids under the names SALCARE SC 80® and SALCARE SC 90®, which are aqueous 30% emulsions of a crosslinked terpolymer of methacrylic acid, of ethyl acrylate and of steareth-10 allyl ether (40/50/10).
(II) polymers comprising at least one hydrophilic unit of unsaturated olefinic carboxylic acid, and at least one hydrophobic unit of (C₁₀-C₃₀)alkyl ester of unsaturated carboxylic acid.

For example, these polymers are chosen from those in which the hydrophilic unit of unsaturated olefinic carboxylic acid corresponds to the monomer of formula (XVI) below: 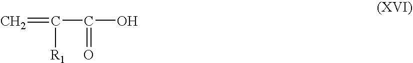
wherein R₁ is chosen from H, CH₃ and C₂H₅, that is to say acrylic acid, methacrylic acid or ethacrylic acid units, and wherein the hydrophobic unit of (C₁₀-C₃₀)alkyl ester of unsaturated carboxylic acid corresponds to the monomer of formula (XVII) below: 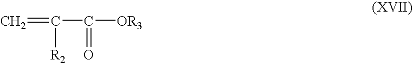
wherein R₂ is chosen from H, CH₃ and C₂H₅ (that is to say acrylate, methacrylate or ethacrylate units) and, for example, H (acrylate units) and CH₃ (methacrylate units), and R₃ is chosen from C₁₀-C₃₀ and, for example, C₁₂-C₂₂ alkyl radicals.

Examples of (C₁₀-C₃₀) alkyl esters of unsaturated carboxylic acids include lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to U.S. Pat. No. 3,915,921 and U.S. Pat. No. 4,509,949.

Examples of anionic associative polymers of this type include polymers formed from a monomer mixture comprising:
(i) essentially acrylic acid,
(ii) an ester of formula (XVII) described above wherein R₂ is chosen from H and CH₃, R₃ is chosen from alkyl radicals comprising from 12 to 22 carbon atoms, and
(iii) a crosslinking agent, which is a well-known copolymerizable unsaturated polyethylenic monomer, for instance diallyl phthalate, allyl(meth)acrylate, divinylbenzene, (poly)ethylene glycol dimethacrylate and methylenebisacrylamide.

Examples of anionic associative polymers of this type include polymers comprising from 60% to 95% by weight of acrylic acid (hydrophilic unit), 4% to 40% by weight of C₁₀-C₃₀ alkyl acrylate (hydrophobic unit) and 0% to 6% by weight of crosslinking polymerizable monomer, or alternatively those comprising from 96% to 98% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described above.

Among the above-polymers, those that may be used include the products sold by the company Goodrich under the trade names PEMULEN TR1®, PEMULEN TR2® and CARBOPOL 1382®, and, further, for example, PEMULEN TR1®, and the product sold by the company SEPPIC under the name COATEX SX®.
(III) maleic anhydride/C₃₀-C₃₈ α-olefin/alkyl maleate terpolymers, such as the product (maleic anhydride/C₃₀-C₃₈ α-olefin/isopropyl maleate copolymer) sold under the name PERFORMA V 1608® by the company Newphase Technologies.
(IV) acrylic terpolymers comprising:
(a) 20% to 70% by weight of a carboxylic acid comprising α,β-monoethylenic unsaturation,
(b) 20% to 80% by weight of a non-surfactant monomer comprising α,β-monoethylenic unsaturation other than (a), and
(c) 0.5% to 60% by weight of a nonionic monourethane which is the product of reaction of a monohydric surfactant with a monoisocyanate comprising monoethylenic unsaturation,
such as those described in Patent Application No. EP-A-0 173 109 and, further, for example, the terpolymer described in Example 3, namely a methacrylic acid/methyl acrylate/behenyl alcohol dimethyl-meta-isopropenylbenzylisocyanate ethoxylated (40 EO) terpolymer, as an aqueous 25% dispersion.
(V) copolymers comprising among their monomers a carboxylic acid comprising α,β-monoethylenic unsaturation and an ester of a carboxylic acid comprising α,β-monoethylenic unsaturation and of an oxyalkylenated fatty alcohol.

For example, these compounds also comprise as a monomer an ester of a carboxylic acid comprising α,β-monoethylenic unsaturation and a C₁-C₄ alcohol.

An example of a compound of this type that may be mentioned is ACULYN 22® sold by the company Rohm & Haas, which is a methacrylic acid/ethyl acrylate/stearyl methacrylate oxyalkylenated terpolymer.

Among the cationic associative polymers of cationic type that may be mentioned:
(I) cationic associative polyurethanes, the family of which has been described in French Patent Application No. 00/09609; it may be represented by the formula (XVIII) below:
   R—X—(P)n-[L-(Y)m]r-L′-(P′)p-X′—R′ (XVIII)
wherein:
   R and R′, which may be identical or different, are chosen from hydrophobic groups and hydrogen atoms;
   X and X′, which may be identical or different, are each chosen from groups comprising at least one amine functional group optionally bearing at least one hydrophobic group, or alternatively a group L″;
   L, L′ and L″, which may be identical or different, are each chosen from groups derived from a diisocyanate;
   P and P′, which may be identical or different, are each chosen from groups comprising an amine functional group optionally bearing at least one hydrophobic group;
   Y is chosen from hydrophilic groups;
   r is an integer ranging from 1 to 100, for example, from 1 to 50 and, further, for example, from 1 to 25;
   n, m and p each range, which may be identical or different, from 0 to 1,000; and
   wherein the molecule comprises at least one functional group chosen from protonated and quaternized amine functional groups and at least one hydrophobic group.

In one embodiment of these polyurethanes, the only hydrophobic groups are the groups R and R′ at the chain ends.

According to one embodiment, the cationic associative polyurethanes are chosen from those corresponding to formula (XVIII) described above and wherein:
R and R′, which may be identical or different, are each chosen from hydrophobic groups;
X and X′, which may be identical or different, are each chosen from groups
n and p, which may be identical or different, are each integers ranging from 1 to 1,000; and
L, L′, L″, P, P′, Y, r and m have the meaning given above.

In another embodiment, the cationic associative polyurethanes are chosen from those corresponding to formula (XVIII) above wherein:
R and R′, which may be identical or different, are each chosen from hydrophobic groups; X and X′, which may be identical or different, are each chosen from groups L″; n and p are 0; and L, L′, L″, Y, r and m have the meaning given above.

The fact that n and p are 0 means that these polymers do not comprise units derived from a monomer comprising an amine functional group, incorporated into the polymer during the polycondensation. The protonated amine functional groups of these polyurethanes result from the hydrolysis of excess isocyanate functional groups, at the chain end, followed by alkylation of the primary amine functional groups formed with alkylating agents comprising a hydrophobic group, i.e. compounds of the type RQ or R′Q, wherein R and R′ are as defined above and Q is chosen from leaving groups, such as a halide, a sulfate, etc.

In yet another embodiment the cationic associative polyurethanes are chosen from those corresponding to formula (Ia) above wherein:
R and R′, which may be identical or different, are each chosen from hydrophobic groups;
X and X′, which may be identical or different, are each chosen from groups comprising at least one quaternary amine;
n and p are equal to zero; and
L, L′, Y, r and m have the meaning given above.

The number-average molecular mass of the cationic associative polyurethanes ranges from 400 to 500,000, for example, from 1,000 to 400,000 and, further, for example, from 1,000 to 300,000.

As used herein, the “hydrophobic group” means a radical or polymer comprising a saturated or unsaturated, linear or branched hydrocarbon-based chain, which may comprise at least one hetero atom, such as P, O, N or S, or a radical comprising a perfluoro or silicone chain. When the hydrophobic group is chosen from hydrocarbon-based radicals, it comprises at least 10 carbon atoms, for example, from 10 to 30 carbon atoms, for example, from 12 to 30 carbon atoms and, further, for example, from 18 to 30 carbon atoms.

In one embodiment, the hydrocarbon-based group is derived from a monofunctional compound.

By way of example, the hydrophobic group may be derived from a fatty alcohol such as stearyl alcohol, dodecyl alcohol or decyl alcohol. It may also be chosen from hydrocarbon-based polymers, such as polybutadiene.

When X and/or X′ are chosen from groups comprising at least one group chosen from tertiary and quaternary amines, X and/or X′ may be chosen from one of the following formulae: 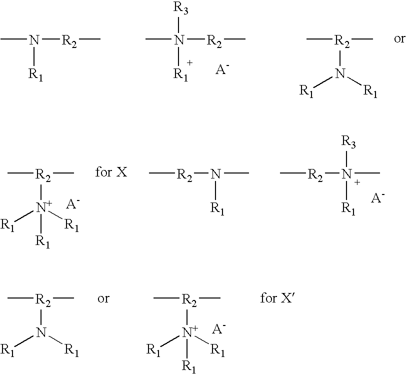
wherein:
   R₂, which may be identical or different, is chosen from linear and branched alkylene radicals comprising from 1 to 20 carbon atoms, optionally comprising at least one ring chosen from saturated and unsaturated rings, and an arylene radical, at least one of the carbon atoms of the alkylene radicals possibly being replaced with a hetero atom chosen from N, S, O and P;
   R₁ and R₃, which may be identical or different, are each chosen from linear and branched C₁-C₃₀ alkyl and alkenyl radicals and an aryl radical, at least one of the carbon atoms possibly being replaced with a hetero atom chosen from N, S, O and P; and
   A⁻ is chosen from physiologically acceptable counterions.

The groups L, L′ and L″ are chosen from groups of formula: 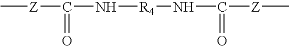
wherein:
   Z is chosen from —O—, —S— and —NH—; and
   R₄ is chosen from linear and branched alkylene radicals comprising from 1 to 20 carbon atoms, optionally comprising at least one ring chosen from saturated and unsaturated rings, and an arylene radical, at least one of the carbon atoms of the alkylene radicals possibly being replaced with a hetero atom chosen from N, S, O and P.

The groups P and P′, which may be identical or different, comprising at least one amine functional group may be chosen from at least one of the following formulae: 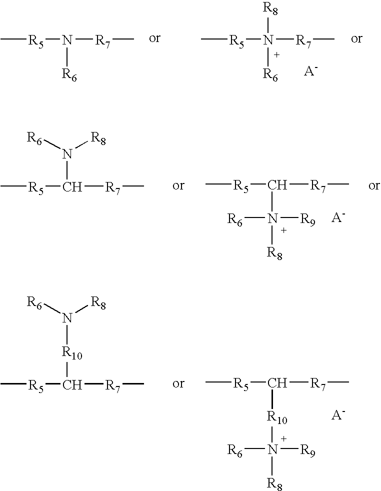
wherein:
   R₅ and R₇ have the same meanings as R₂ defined above;
   R₆, R₈ and R₉ have the same meanings as R₁ and R₃ defined above;
   R₁₀ is chosen from linear and branched, optionally unsaturated alkylene groups optionally comprising at least one hetero atom chosen from N, O, S and P; and
   A⁻ is chosen from physiologically acceptable counterions.

With regard to the meaning of Y, the term “hydrophilic group” means a polymeric or non-polymeric water-soluble group.

By way of example, when Y is not a polymer, mention may be made of ethylene glycol, diethylene glycol and propylene glycol.

When Y is a hydrophilic polymer, it may be chosen, for example, from at least one of polyethers, sulfonated polyesters, and sulfonated polyamides. The hydrophilic compound may, for example, be a polyether and, for example, a poly(ethylene oxide) or poly(propylene oxide).

The cationic associative polyurethanes of formula (XVIII) that may be used are formed from diisocyanates and from various compounds with functional groups comprising labile hydrogen. The functional groups comprising labile hydrogen may be chosen from alcohol, primary and secondary amine and thiol functional groups, forming, after reaction with diisocyanate functional groups, polyurethanes, polyureas and polythioureas, respectively. As used herein, the expression “polyurethanes” encompasses these three types of polymer, for example, polyurethanes per se, polyureas and polythioureas, and also copolymers thereof.

A first type of compound involved in the preparation of the polyurethane of formula (XVIII) is a compound comprising at least one unit comprising an amine functional group. This compound may be multifunctional, but the compound may, for example, be difunctional, that is to say that, according to one embodiment, this compound comprises two labile hydrogen atoms borne, for example, by a hydroxyl, primary amine, secondary amine or thiol functional groups. A mixture of multifunctional and difunctional compounds, in which the percentage of multifunctional compounds is low, may also be used.

As mentioned above, this compound may comprise more than one unit comprising an amine functional group. In this case, it is a polymer bearing a repetition of the unit comprising an amine functional group.

Compounds of this type may be chosen from one of the following formulae:
HZ-(P)n-ZH
and
HZ-(P′)p-ZH
wherein Z, P, P′, n and p are as defined above.

Examples of compounds comprising an amine functional group that may be mentioned include N-methyldiethanolamine, N-tert-butyldiethanolamine and N-sulfoethyldiethanolamine.

The second compound involved in the preparation of the polyurethane of formula (XVIII) is a diisocyanate corresponding to the formula:
O═C═N—R₄—N═C═O
wherein R₄ is as defined above.

By way of example, mention may be made of methylenediphenyl diisocyanate, methylenecyclohexane diisocyanate, isophorone diisocyanate, toluene diisocyanate, naphthalene diisocyanate, butane diisocyanate, and hexane diisocyanate.

A third compound involved in the preparation of the polyurethane of formula (XVIII) is a hydrophobic compound intended to form the terminal hydrophobic groups of the polymer of formula (XVIII).

This compound comprises a hydrophobic group and a functional group comprising labile hydrogen, for example, a hydroxyl, primary or secondary amine, or thiol functional groups.

For example, this compound may be a fatty alcohol chosen from stearyl alcohol, dodecyl alcohol, and decyl alcohol. When this compound comprises a polymeric chain, it may be, for example, α-hydroxylated hydrogenated polybutadiene.

The hydrophobic group of the polyurethane of formula (XVIII) may also result from the quaternization reaction of the tertiary amine of the compound comprising at least one tertiary amine unit. Thus, the hydrophobic group is introduced via the quaternizing agent. This quaternizing agent is a compound chosen from RQ and R′Q, wherein R and R′ are as defined above and Q is chosen from leaving groups such as a halide, a sulfate, etc.

The cationic associative polyurethane may also comprise a hydrophilic block. This block is provided by a fourth type of compound involved in the preparation of the polymer. This compound may be multifunctional, for example, difunctional. It is also possible to have a mixture in which the percentage of multifunctional compound is low.

The functional groups comprising labile hydrogen are chosen from alcohol, primary and secondary amine and thiol functional groups. This compound may be a polymer terminated at the chain ends with one of these functional groups comprising labile hydrogen.

By way of example, when the hydrophilic compound is not a polymer, mention may be made of ethylene glycol, diethylene glycol and propylene glycol.

When it is a hydrophilic polymer, mention may be made, for example, of at least one of polyethers, sulfonated polyesters and sulfonated polyamides. The hydrophilic compound may, for example, be polyether and, further, for example, poly(ethylene oxide) or poly(propylene oxide).

The hydrophilic group termed Y in formula (XVIII) is optional. For example, the units comprising a quaternary amine or protonated functional group may suffice to provide the solubility or water-dispersibility required for this type of polymer in an aqueous solution.

Although the presence of a hydrophilic group Y is optional, cationic associative polyurethanes comprising such a group may, for example, be used.
(II) quaternized cellulose derivatives and polyacrylates comprising non-cyclic amine side groups.

The quaternized cellulose derivatives may, for example, be chosen from:
quaternized celluloses modified with groups comprising at least one fatty chain, such as alkyl, arylalkyl and alkylaryl groups comprising at least 8 carbon atoms and
quaternized hydroxyethylcelluloses modified with groups comprising at least one fatty chain, such as alkyl, arylalkyl and alkylaryl groups comprising at least 8 carbon atoms.

The alkyl radicals borne by the above quaternized celluloses or hydroxyethylcelluloses comprises, for example, from 8 to 30 carbon atoms. The aryl radicals are chosen, for example, from phenyl, benzyl, naphthyl and anthryl groups.

Examples of quaternized alkylhydroxyethylcelluloses comprising C₈-C₃₀ fatty chains that may be mentioned include the products QUATRISOFT LM 200®, QUATRISOFT LM-X 529-18-A®, QUATRISOFT LM-X 529-18B® (C₁₂ alkyl) and QUATRISOFT LM-X 529-8® (C₁₈ alkyl) sold by the company Amerchol, and the products CRODACEL QM®, CRODACEL QL® (C₁₂ alkyl) and CRODACEL QS® (C₁₈ alkyl) sold by the company Croda.

The amphoteric associative polymers are, for example, chosen from those comprising at least one non-cyclic cationic unit. In one embodiment, the amphoteric associative polymers that are preferred are those prepared from or comprising 1 to 20 mol %, for example, 1.5 to 15 mol % and, further, for example, from 1.5 to 6 mol % of fatty-chain monomer relative to the total number of moles of monomers.

The amphoteric associative polymers that may be used comprise or are prepared by copolymerizing:
1) at least one monomer of formula (XIX) or (XX): 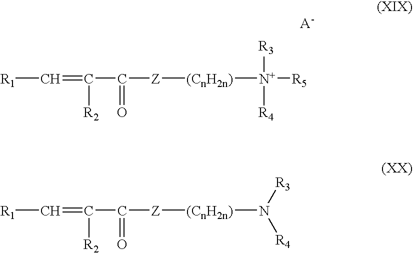
wherein R₁ and R₂, which may be identical or different, are each chosen from a hydrogen atom and a methyl radical, R₃, R₄ and R₅, which may be identical or different, are each chosen from linear and branched alkyl radicals comprising from 1 to 30 carbon atoms;
   Z is chosen from an NH group and an oxygen atom;
   n is an integer ranging from 2 to 5; and
   A⁻ is chosen from anions derived from an organic or mineral acid, such as a methosulfate anion or a halide, such as chloride or bromide;
2) at least one monomer of formula (XXI)
   R₆—CH═CR₇—COOH (XXI)
wherein R₆ and R₇, which may be identical or different, are each chosen from hydrogen atoms and methyl radicals; and
3) at least one monomer of formula (XXII):
   R₆—CH═CR₇—COXR₈ (XXII)
wherein R₆ and R₇, which may be identical or different, are each chosen from hydrogen atoms and methyl radicals, X is chosen from an oxygen and a nitrogen atom and R₈ is chosen from linear and branched alkyl radicals comprising from 1 to 30 carbon atoms; wherein
at least one of the monomers of formula (XIX), (XX) or (XXII) comprises at least one fatty chain.

The monomers of formulae (XIX) and (XX) may be chosen from the following:
dimethylaminoethyl methacrylate, dimethylaminoethyl acrylate,
diethylaminoethyl methacrylate, diethylaminoethyl acrylate,
dimethylaminopropyl methacrylate, dimethylaminopropyl acrylate,
dimethylaminopropylmethacrylamide, and dimethylaminopropylacrylamide,
these monomers optionally being quaternized, for example, with a C₁-C₄ alkyl halide or a C₁-C₄ dialkyl sulfate.

In one embodiment, the monomer of formula (XIX) is chosen from acrylamidopropyltrimethylammonium chloride and methacrylamidopropyl-trimethylammonium chloride.

The monomers of formula (XXI) may, for example, be chosen from acrylic acid, methacrylic acid, crotonic acid, and 2-methylcrotonic acid. In one embodiment, the monomer of formula (XXI) is acrylic acid.

The monomers of formula (XXII) may, for example, be chosen from the group comprising C₁₂-C₂₂ and, for example, C₁₆-C₁₈ alkyl acrylates and methacrylates.

The monomers constituting the fatty-chain amphoteric polymers of the invention may, for example, be already neutralized and/or quaternized.

The ratio of the number of cationic charges/anionic charges may, for example, be equal to 1.

The amphoteric associative polymers disclosed herein may comprise from 1 mol % to 10 mol % of the monomer comprising a fatty chain (monomer of formula (XIX), (XX) or (XXII)), and, for example, from 1.5 mol % to 6 mol %.

The weight-average molecular weights of the amphoteric associative polymers disclosed herein may range from 500 to 50,000,000 and, further, for example, from 10 000 to 5,000,000.

The amphoteric associative polymers disclosed herein may also comprise other monomers such as nonionic monomers and, for example, C₁-C₄ alkyl acrylates and methacrylates.

Amphoteric associative polymers disclosed herein are described and prepared, for example, in Patent Application No. WO 98/44012.

For example, the amphoteric associative polymers may be chosen from acrylic acid/(meth)acrylamidopropyltrimethylammon ium chloride/stearyl methacrylate terpolymers.

The non-ionic associative polymers that may be used may, for example, be chosen from:
(1) celluloses modified with groups comprising at least one fatty chain; examples that may be mentioned include:
   hydroxyethylcelluloses modified with groups comprising at least one fatty chain, such as alkyl, arylalkyl or alkylaryl groups, and in which the alkyl groups are, for example, C₈-C₂₂ alkyl groups, for instance the product NATROSOL PLUS GRADE 330 CS® (C₁₆ alkyls) sold by the company Aqualon, or the product BERMOCOLL EHM 100® sold by the company Berol Nobel,
   polymers modified with alkylphenyl polyalkylene glycol ether groups, such as the product AMERCELL POLYMER HM-1500® (nonylphenyl polyethylene glycol (15) ether) sold by the company Amerchol.
(2) hydroxypropyl guars modified with groups comprising at least one fatty chain, such as the product ESAFLOR HM 22® (C₂₂ alkyl chain) sold by the company Lamberti, and the products RE210-18® (C₁₄ alkyl chain) and RE205-1® (C₂₀ alkyl chain) sold by the company Rhone-Poulenc.
(3) copolymers of vinylpyrrolidone and of fatty-chain hydrophobic monomers; examples that may be mentioned include:
   the products ANTARON V216® or GANEX V216® (vinylpyrrolidone/hexadecene copolymer) sold by the company I.S.P.
   the products ANTARON V220® or GANEX V220® (vinylpyrrolidone/eicosene copolymer) sold by the company I.S.P.
(4) copolymers of C₁-C₆ alkyl methacrylates or acrylates and of amphiphilic monomers comprising at least one fatty chain, such as the oxyethylenated methyl acrylate/stearyl acrylate copolymer sold by the company Goldschmidt under the name ANTIL 208®.
(5) copolymers of hydrophilic methacrylates or acrylates and of hydrophobic monomers comprising at least one fatty chain, such as the polyethylene glycol methacrylate/lauryl methacrylate copolymer.
(6) polyurethane polyethers comprising in their chain both hydrophilic blocks usually of polyoxyethylenated nature and hydrophobic blocks which may be aliphatic sequences alone and/or cycloaliphatic and/or aromatic sequences.
(7) polymers with an aminoplast ether skeleton comprising at least one fatty chain, such as the PURE THIX® compounds sold by the company Sud-Chemie.

For example, the polyurethane polyethers comprise at least two hydrocarbon-based lipophilic chains comprising from 6 to 30 carbon atoms, separated by a hydrophilic block, the hydrocarbon-based chains possibly being pendent chains, or chains at the end of the hydrophilic block. For example, it is possible for at least one pendent chain to be included. In addition, the polymer may comprise a hydrocarbon-based chain at one end or at both ends of a hydrophilic block.

The polyurethane polyethers may be multiblock, such as in triblock form. Hydrophobic blocks may be at each end of the chain (for example: triblock copolymer with a hydrophilic central block) or distributed both at the ends and in the chain (for example: multiblock copolymer). These same polymers may also be graft polymers or starburst polymers.

The nonionic fatty-chain polyurethane polyethers may be triblock copolymers in which the hydrophilic block is a polyoxyethylenated chain comprising from 50 to 1,000 oxyethylene groups. The nonionic polyurethane polyethers comprise a urethane linkage between the hydrophilic blocks, whence arises the name.

By extension, also included among the nonionic fatty-chain polyurethane polyethers are those in which the hydrophilic blocks are linked to the lipophilic blocks via other chemical bonds.

Examples of nonionic fatty-chain polyurethane polyethers that may be used include RHEOLATE 205® comprising a urea functional group, sold by the company Rheox, or RHEOLATE® 208, 204 or 212, and also ACRYSOL RM 184®.

Mention may also be made of the product ELFACOS T210® comprising a C₁₂₋₁₄ alkyl chain, and the product ELFACOS T212® comprising a C₁₈ alkyl chain, from Akzo.

The product DW 1206B® from Rohm & Haas comprising a C₂₀ alkyl chain and a urethane linkage, sold at a solids content of 20% in water, may also be used.

It is also possible to use solutions or dispersions of these polymers, for example, in water or in aqueous-alcoholic medium. Examples of such polymers that may be mentioned are RHEOLATE® 255, RHEOLATE® 278 and RHEOLATE® 244 sold by the company Rheox. The products DW 1206F and DW 1206J sold by the company Rohm & Haas may also be used.

The polyurethane polyethers that may be used include those described in the article by G. Formum, J. Bakke and Fk. Hansen—Colloid Polym. Sci 271, 380.389 (1993).

In one embodiment, it is possible to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold, for example, by the company Rohm & Haas under the names ACULYN 44® and ACULYN 46® [ACULYN 46® is a polycondensate of polyethylene glycol comprising 150 or 180 mol of ethylene oxide, of stearyl alcohol and of methylenebis(4-cyclohexyl isocyanate) (SMDI), at 15% by weight in a matrix of maltodextrin (4%) and water (81%); ACULYN 44® is a polycondensate of polyethylene glycol comprising 150 or 180 mol of ethylene oxide, of decyl alcohol and of methylenebis(4-cyclohexylisocyanate) (SMDI), at 35% by weight in a mixture of propylene glycol (39%) and water (26%)].

Non-associative gelling agents may also be used as thickeners; included among these are polymers or copolymers of unsaturated carboxylic organic acids or of unsaturated esters, polysaccharide derivatives, gums, colloidal silicates, polyethylene glycols, polyvinyl pyrrolidones and hydrophilic silica gels.

The at least one thickener may be present in the at least one styling composition disclosed herein in an amount ranging from 0.01% to 10% by weight and, further, for example, from 0.1% to 5% by weight, relative to the total weight of the composition.

According to one embodiment, the at least one styling composition disclosed herein also comprises at least one surfactant.

The at least one surfactant may be chosen from:

(i) Anionic Surfactant(s):

By way of example of anionic surfactants that can be used, alone or as mixtures mention may be made, for example, of at least one (non-limiting list) of salts (such as alkali metal salts, for example, sodium salts, ammonium salts, amine salts, amino alcohol salts and magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; (C₆-C₂₄)alkyl sulfosuccinates, (C₆-C₂₄)alkyl ether sulfosuccinates, (C₆-C₂₄)alkylamide sulfosuccinates; (C₆-C₂₄)alkyl sulfoacetates; (C₆-C₂₄)acyl sarcosinates; and (C₆-C₂₄)acyl glutamates. It is also possible to use (C₆-C₂₄)alkylpolyglycoside carboxylic esters such as alkylglucoside citrates, alkylpolyglycoside tartrates and alkylpolyglycoside sulfosuccinates, alkylsulfosuccinamates; acyl isethionates and N-acyl taurates, wherein the alkyl or acyl radical of all of these different compounds comprises, for example, from 12 to 20 carbon atoms and the aryl radical is chosen, for example, from phenyl and benzyl groups. Among the anionic surfactants which can also be used, mention may also be made of fatty acid salts, such as oleic, ricinoleic, palmitic and stearic acid salts; coconut oil acid and hydrogenated coconut oil acid; and acyl lactylates, wherein the acyl radical comprises 8 to 20 carbon atoms. It is also possible to use alkyl D-galactoside uronic acids and salts thereof, polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and salts thereof, such as those comprising from 2 to 50 alkylene oxide groups, for example, ethylene oxide groups.

(ii) Nonionic Surfactant(s):

The nonionic surfactants are, themselves also, compounds that are well known per se (see, for example, “Handbook of Surfactants” by M. R. Porter, published by Blackie & Son (Glasgow and London), 1991, pp. 116-178) and their nature is not a critical factor in the context of the present disclosure. Thus, the non-ionic surfactants can be chosen, for example, from (non-limiting list) polyethoxylated and polypropoxylated, alkylphenols, alpha-diols and alcohols, comprising at least one fatty chain comprising, for example, 8 to 18 carbon atoms, it being possible for the number of ethylene oxide or propylene oxide groups to range, for example, from 2 to 50. Mention may also be made of copolymers of ethylene oxide and of propylene oxide, condensates of ethylene oxide and of propylene oxide with fatty alcohols; polyethoxylated fatty amides, for example, comprising from 2 to 30 mol of ethylene oxide, polyglycerolated fatty amides comprising on average 1 to 5, and, for example, 1.5 to 4, glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, and amine oxides such as (C₁₀-C₁₄)alkylamine oxides and N-acylaminopropylmorpholine oxides.

(iii) Amphoteric or Zwitterionic Surfactant(s):

The amphoteric or zwitterionic surfactants, the nature of which is not a critical factor in the context of the present disclosure may, for example, be chosen from (non-limiting list), aliphatic secondary and tertiary amine derivatives, wherein the aliphatic radical is chosen from linear and branched chains comprising from 8 to 18 carbon atoms and comprising at least one water-solubilizing anionic group (for example, carboxylate, sulfonate, sulfate, phosphate and phosphonate); mention may also be made of (C₈-C₂₀)alkylbetaines, sulfobetaines, (C₈-C₂₀)alkylamido(C₁-C₆)alkylbetaines, and (C₈-C₂₀)alkylamido(C₁-C₆)alkylsulfobetaines.

Among the amine derivatives, mention may be made of the products sold under the name Miranol, as described in U.S. Pat. Nos. 2,528,378 and 2,781,354 and classified in the CTFA dictionary, 3rd edition, 1982, under the names Amphocarboxyglycinates and Amphocarboxypropionates, with the respective structures:
R₂—CONHCH₂CH₂—N(R₃)(R₄)(CH₂COO⁻)
wherein: R₂ is chosen from alkyl radicals of an acid R₂—COOH present in hydrolyzed coconut oil and heptyl, nonyl, and undecyl radicals, R₃ is chosen from beta-hydroxyethyl groups, and R₄ is chosen from carboxymethyl groups; and
   R₂′—CONHCH₂CH₂—N(B)(C)
wherein:
   B is —CH₂CH₂OX′, C is —(CH₂)ₙ—Y′, wherein z=1 or 2;
   X′ is chosen from a —CH₂CH₂—COOH group and a hydrogen atom;
   Y′ is chosen from —COOH and a —CH₂—CHOH—SO₃H radical; and
   R₂′ is chosen from alkyl radicals of an acid R₉—COOH present in coconut oil or in hydrolyzed linseed oil, alkyl radicals, for example, C₇, C₉, C₁₁ and C₁₃ alkyl radicals, a C₁₇ alkyl radical and iso forms thereof, and an unsaturated C₁₇ radical.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid, and Cocoamphodipropionic acid.

For example, mention may be made of the cocoamphodiacetate sold under the trade name MIRANOL® C2M concentrate by the company Rhodia Chimie.

(iv) Cationic Surfactants:

Among the cationic surfactants, mention may be made, for example, (non-limiting list) of: salts of optionally polyoxyalkylenated primary, secondary and tertiary fatty amines; quaternary ammonium salts such as tetraalkylammonium, alkylamidoalkyltrialkylammonium, trialkylbenzylammonium, trialkylhydroxyalkylammonium and alkylpyridinium chlorides and bromides; imidazoline derivatives; and amine oxides of cationic nature.

The at least one surfactant may be present in an amount ranging from 0.01% to 40% and, for example, from 0.1% to 30% by weight, relative to the total weight of the composition.

Further disclosed herein is a cosmetic process for shaping and/or holding the hairstyle, comprising vaporizing onto the hair the contents of the aerosol device disclosed herein.

When the contents of the aerosol device are vaporized onto the hair, the aerosol device disclosed herein may generate a spray or a mousse.

The invention is illustrated in greater detail by the examples described below. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters in the disclosure above are approximations, the numerical values set forth in the specific examples are reported as precisely as possible. Any numerical value, however, inherently contains certain errors necessarily resulting from the standard deviation found in its respective testing measurements.

### EXAMPLE 1

### Preparation of a Poly(Acrylic Acid/Methyl Acrylate) Pseudo-Block Polymer

100 g of butyl acetate were introduced into a 1 liter reactor and the temperature was then increased so as to go from room temperature (25° C.) to 90° C. in 1 hour.

30 g of acrylic acid, 30 g of methyl acrylate, 40 g of butyl acetate, 70 g of isopropanol and 1.8 g of 2,5-bis(2-ethylhexanoylperoxy)-2,5-dimethylhexane (TRIGONOX® 141 from Akzo Nobel) were then added, at 90° C. and over 1 hour.

The mixture was maintained at 90° C. for 1 hour.

90 g of methyl acrylate, 70 g of butyl acetate, 20 g of isopropanol and 1.2 g of 2,5-bis(2-ethylhexanoylperoxy)-2,5-dimethylhexane were then introduced into the above mixture, still at 90° C. and over 1 hour.

The mixture was maintained at 90° C. for 3 hours, it was then diluted with 105 g of butyl acetate and 45 g of isopropanol, and the mixture was then cooled.

A solution containing 40% polymer active material in a butyl acetate/isopropanol mixture was obtained.

A polymer comprising a first poly(acrylic acid/methyl acrylate) block with a Tg of 80° C., a second polymethyl acrylate block with a Tg of 10° C. and an intermediate segment that was an acrylic acid/methyl acrylate/polymethyl acrylate random polymer was obtained.

This polymer had a weight-average mass of 50,000 g/mol and a number-average mass of 17,000, i.e. a polydispersity index I of 2.95.

It had a glass transition temperature (Tg) of 49° C.

### EXAMPLE 2

A styling composition propelled with a liquefied gas in a standard aerosol device was prepared.

The proportions of the various ingredients, given as weight percentages relative to the total contents of the aerosol device, are given in the table below.

| Poly(acrylic acid/methyl acrylate) pseudo-block polymer | 3% AM |
|---|---|
| Mexomer PW (100% neutralized) | 3% AM |
| Ethanol | 5% AM |
| Demineralized water | 54% |
| Dimethyl ether | 35% |

### EXAMPLE 3

A styling mousse was prepared:

The proportions of the various ingredients, given as weight percentages relative to the total contents of the aerosol device, are given in the table below.

| Poly(acrylic acid/methyl acrylate) pseudo-block polymer | 2% AM |
|---|---|
| Oxyethylenated sorbitan monolaurate | 0.2% AM |
| Demineralized water | 92.8% |
| Isobutane/propane (85/15) | 5% |

### EXAMPLE 4

A styling composition propelled with compressed air was prepared.

The proportions of the various ingredients, given as weight percentages relative to the total contents of the aerosol device, are given in the table below.

| Poly(acrylic acid/methyl acrylate) pseudo-block polymer | 3% AM |
|---|---|
| Mexomer PW (100% neutralized) | 3% AM |
| Ethanol | 5% AM |
| Demineralized water | 89% |

Weight percentage data

This styling composition was packaged in a pocket aerosol device. This device is sold under the name EP SPRAY, and comprises an assembly comprising a pocket hermetically welded to a valve and a turbulent-nozzle diffuser. The valve was fixed onto a standard aerosol can.

The pocket was filled with the composition of Example 4 and compressed air was introduced between the pocket and the aerosol can at a sufficient pressure to cause the product to exit in the form of a spray.

The pressure of the compressed gas ranged from 1 to 12 bar and, for example, from 9 to 11 bar.

1. An aerosol device comprising,
at least one propellant and
at least one styling composition comprising, in a cosmetically acceptable medium comprising more than 50% by weight of water relative to the total weight of the composition, at least one pseudo-block polymer and at least one additional fixing polymer, wherein the at least one pseudo-block polymer comprises at least one first block and at least one second block that are mutually incompatible and that have different glass transition temperatures (Tg), wherein the at least one first and at least one second block are linked together via an intermediate segment comprising at least one constituent monomer of the at least one first block and at least one constituent monomer of the at least one second block and wherein the at least one pseudo-block polymer has a polydispersity index (I) of greater than 2.

2. The device according to claim 1, wherein the cosmetically acceptable medium of the at least one styling composition comprises more than 70% by weight of water, relative to the total weight of the composition.

3. The device according to claim 1, wherein the at least one first block of the at least one pseudo-block polymer is chosen from:
a) a block with a Tg of greater than or equal to 40° C.;
b) a block with a Tg of less than or equal to 20° C.;
c) a block with a Tg ranging from 20 to 40° C., and
the at least one second block is chosen from a category a), b) and c) different from the at least one first block.

4. The device according to claim 3, wherein the block with a Tg of greater than or equal to 40° C. is totally or partially derived from at least one monomer which is such that the homopolymer prepared from the at least one monomer has a glass transition temperature of greater than or equal to 40° C.

5. The device according to claim 4, wherein the at least one pseudo-block polymer is such that the at least one monomer whose corresponding homopolymer has a glass transition temperature of greater than or equal to 40° C. is chosen from the following monomers:
methacrylates of formula CH₂═C(CH₃)—COOR₁, wherein R₁ is chosen from linear and branched unsubstituted alkyl groups comprising from 1 to 4 carbon atoms;
acrylates of formula CH₂═CH—COOR₂, wherein R₂ is chosen from C₄ to C₁₂ cycloalkyl groups; and
(meth)acrylamides of formula: 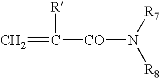
wherein R₇ and R₈, which may be identical or different, are each chosen from hydrogen atoms and linear and branched alkyl groups comprising 1 to 12 carbon atoms; or R₇ is H and R₈ is a 1,1-dimethyl-3-oxobutyl group, and R′ is chosen from H and methyl.

6. The device according to claim 5, wherein the at least one pseudo-block polymer is such that the at least one monomer whose corresponding homopolymer has a glass transition temperature of greater than or equal to 40° C. is chosen from methyl methacrylate, isobutyl methacrylate, and isobornyl(meth)acrylate.

7. The device according to claim 3, wherein the block with a Tg of less than or equal to 20° C. is totally or partially derived from at least one monomer which is such that the homopolymer prepared from the at least one monomer has a glass transition temperature of less than or equal to 20° C.

8. The device according to claim 7, wherein the at least one monomer whose corresponding homopolymer has a glass transition temperature of less than or equal to 20° C. are chosen from the following monomers:
acrylates of formula CH₂═CHCOOR₃, wherein R₃ is chosen from linear and branched C₁ to C₁₂ unsubstituted alkyl groups, with the exception of the tert-butyl group, wherein at least one hetero atom chosen from O, N and S is optionally intercalated;
methacrylates of formula CH₂═C(CH₃)—COOR₄, wherein R₄ is chosen from linear and branched C₆ to C₁₂ unsubstituted alkyl groups, wherein at least one hetero atom chosen from O, N and S is optionally intercalated;
vinyl esters of formula R₅—CO—O—CH═CH₂ wherein R₅ is chosen from linear and branched C₄ to C₁₂ alkyl groups;
C₄ to C₁₂ alkyl vinyl ethers; and
N—(C₄ to C₁₂)alkyl acrylamides.

9. The device according to claim 7, wherein the at least one pseudo-block polymer is such that the at least one monomer whose corresponding homopolymer has a glass transition temperature of less than or equal to 20° C. is chosen from alkyl acrylates whose alkyl chain comprises from 1 to 10 carbon atoms, with the exception of a tert-butyl group.

10. The device according to claim 3, wherein the block with a Tg ranging from 20 to 40° C. is totally or partially derived from at least one monomer which is such that the homopolymer prepared from the at least one monomer has a glass transition temperature ranging from 20 to 40° C.

11. The device according to claim 10, wherein the block with a Tg ranging from 20 to 40° C. is totally or partially derived from at least one monomer which is such that the corresponding homopolymer has a Tg of greater than or equal to 40° C. and from at least one monomer which is such that the corresponding homopolymer has a Tg of less than or equal to 20° C.

12. The device according to claim 10, wherein the block with a Tg ranging from 20 to 40° C. is totally or partially derived from at least one monomer chosen from methyl methacrylate, isobornyl acrylate and methacrylate, trifluoroethyl methacrylate, butyl acrylate, and 2-ethylhexyl acrylate.

13. The device according to claim 1, comprising at least one propellant and at least one styling composition comprising, in a cosmetically acceptable medium comprising more than 50% by weight of water relative to the total weight of the at least one styling composition, at least one pseudo-block polymer and at least one additional polymer, wherein the at least one pseudo-block polymer comprises at least one first block and at least one second block that are mutually incompatible, the at least one first block having a glass transition temperature (Tg) of greater than or equal to 40° C. and the at least one second block having a glass transition temperature of less than or equal to 20° C., wherein the at least one first and at least one second blocks are linked together via an intermediate segment comprising at least one constituent monomer of the at least one first block and at least one constituent monomer of the at least one second block and wherein the at least one pseudo-block polymer has a polydispersity index (I) of greater than 2.

14. The aerosol device according to claim 13, wherein the at least one first block of the at least one pseudo-block polymer is totally or partially derived from at least one monomer which is such that the homopolymer prepared from the at least one monomer has a glass transition temperature of greater than or equal to 40° C.

15. The aerosol device according to claim 13, wherein the at least one first block of the at least one pseudo-block polymer is a copolymer derived from monomers which are such that the homopolymer prepared from these monomers has a glass transition temperature of greater than or equal to 40° C.

16. The aerosol device according to claim 13, wherein the at least one pseudo-block polymer is such that the at least one monomer whose corresponding homopolymer has a glass transition temperature of greater than or equal to 40° C. is chosen from the following monomers:
methacrylates of formula CH₂═C(CH₃)—COOR₁, wherein R₁ is chosen from linear and branched unsubstituted alkyl groups comprising from 1 to 4 carbon atoms, or R₁ is chosen from C₄ to C₁₂ cycloalkyl groups;
acrylates of formula CH₂═CH—COOR₂, wherein R₂ is chosen from C₄ to C₁₂ cycloalkyl groups;
(meth)acrylamides of formula: 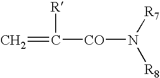
wherein: R₇ and R₈, which may be identical or different, are each chosen from hydrogen atoms and linear and branched alkyl groups comprising from 1 to 12 carbon atoms; or R₇ is H and R₈ is a 1,1-dimethyl-3-oxobutyl group, and R′ is chosen from H and methyl.

17. The aerosol device according to claim 13, wherein the at least one pseudo-block polymer is such that the at least one monomer whose corresponding homopolymer has a glass transition temperature of greater than or equal to 40° C. is chosen from methyl methacrylate, isobutyl methacrylate and isobornyl(meth)acrylate.

18. The aerosol device according to claim 13, wherein the proportion of the at least one first block ranges from 20% to 90% by weight of the polymer.

19. The aerosol device according to claim 13, wherein the at least one pseudo-block polymer is such that the at least one second block is totally or partially derived from at least one monomer which is such that the homopolymer prepared from the at least one monomer has a glass transition temperature of less than or equal to 20° C.

20. The aerosol device according to claim 19, wherein the at least one pseudo-block polymer is such that the at least one second block is a homopolymer derived from at least one monomer which is such that the homopolymer prepared from the at least one monomer has a glass transition temperature of less than or equal to 20° C.

21. The aerosol device according to claim 19, wherein the at least one pseudo-block polymer is such that the at least one monomer whose corresponding homopolymer has a glass transition temperature of less than or equal to 20° C. is chosen from the following monomers:
acrylates of formula CH₂═CHCOOR₃, wherein R₃ is chosen from linear and branched C₁ to C₁₂ unsubstituted alkyl groups, with the exception of the tert-butyl group, wherein at least one hetero atom chosen from O, N and S is optionally intercalated;
methacrylates of formula CH₂═C(CH₃)—COOR₄, wherein R₄ is chosen from linear and branched C₆ to C₁₂ unsubstituted alkyl groups, wherein at least one hetero atom chosen from O, N and S is optionally intercalated;
vinyl esters of formula R₅—CO—O—CH═CH₂, wherein R₅ is chosen from linear and branched C₄ to C₁₂ alkyl groups;
C₄ to C₁₂ alkyl vinyl ethers;
N—(C₄ to C₁₂)alkyl acrylamides.

22. The aerosol device according to claim 19, wherein the at least one pseudo-block polymer is such that the at least one monomer whose corresponding homopolymer has a glass transition temperature of less than or equal to 20° C. is chosen from alkyl acrylates whose alkyl chain comprises from 1 to 10 carbon atoms, with the exception of the tert-butyl group.

23. The aerosol device according claim 19, wherein the proportion of the at least one second block with a Tg of less than or equal to 20° C. ranges from 5% to 75%.

24. The device according to claim 1, comprising at least one propellant and at least one styling composition comprising, in a cosmetically acceptable medium comprising more than 50% by weight of water relative to the total weight of the at least one styling composition, at least one pseudo-block polymer and at least one additional fixing polymer, the at least one pseudo-block polymer comprising at least one first block and at least one second block that are mutually incompatible, the at least one first block having a glass transition temperature (Tg) ranging from 20 to 40° C. and the at least one second block having a glass transition temperature of less than or equal to 20° C. or a glass transition temperature of greater than or equal to 40° C., wherein the at least one first and at least one second block are linked together via an intermediate segment comprising at least one constituent monomer of the at least one first block and at least one constituent monomer of the at least one second block and wherein the at least one pseudo-block polymer has a polydispersity index (I) of greater than 2.

25. The aerosol device according to claim 24, wherein the at least one pseudo-block polymer is such that the at least one first block with a Tg ranging from 20 to 40° C. is totally or partially derived from at least one monomer which is such that the homopolymer prepared from the at least one monomer has a glass transition temperature ranging from 20 to 40° C.

26. The aerosol device according to claim 24, wherein the at least one pseudo-block polymer is such that the at least one first block with a Tg ranging from 20 to 40° C. is a copolymer derived from at least one monomer which is such that the corresponding homopolymer has a Tg of greater than or equal to 40° C. and from at least one monomer which is such that the corresponding homopolymer has a Tg of less than or equal to 20° C.

27. The aerosol device according to claim 24, wherein the at least one pseudo-block polymer is such that the at least one first block with a Tg ranging from 20 to 40° C. is derived from monomers chosen from methyl methacrylate, isobornyl acrylate and methacrylate, butyl acrylate, and 2-ethylhexyl acrylate.

28. The aerosol device according to claim 24, wherein the at least one pseudo-block polymer is such that the proportion of the at least one first block with a Tg ranging from 20 to 40° C. ranges from 10% to 85%.

29. The aerosol device according to claim 24, wherein the at least one pseudo-block polymer is such that the at least one second block with a Tg of greater than or equal to 40° C. is totally or partially derived from at least one monomer which is such that the homopolymer prepared from the at least one monomer has a glass transition temperature of greater than or equal to 40° C.

30. The aerosol device according to claim 24, wherein the at least one pseudo-block polymer is such that the at least one second block has a Tg of greater than or equal to 40° C. and is a homopolymer derived from monomers which are such that the homopolymer prepared from these monomers has a glass transition temperature of greater than or equal to 40° C.

31. The aerosol device according to claim 29, wherein the at least one pseudo-block polymer is such that the at least one monomer whose corresponding homopolymer has a glass transition temperature of greater than or equal to 40° C. is chosen from the following monomers:
methacrylates of formula CH₂═C(CH₃)—COOR₁, wherein R₁ is chosen from linear and branched unsubstituted alkyl groups comprising from 1 to 4 carbon atoms, or R₁ is chosen from C₄ to C₁₂ cycloalkyl groups;
acrylates of formula CH₂═CH—COOR₂, wherein R₂ is chosen from C₄ to C₁₂ cycloalkyl groups;
(meth)acrylamides of formula: 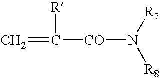
wherein: R₇ and R₈, which may be identical or different, are each chosen from hydrogen atoms and linear and branched alkyl groups comprising 1 to 12 carbon atoms or R₇ is H and R₈ is a 1,1-dimethyl-3-oxobutyl group, and R′ is chosen from H and methyl.

32. The aerosol device according to claim 29, wherein the at least one pseudo-block polymer is such that the at least one monomer whose corresponding homopolymer has a glass transition temperature of greater than or equal to 40° C. is chosen from methyl methacrylate, isobutyl methacrylate, and isobornyl(meth)acrylate.

33. The aerosol device according to claim 29, wherein the at least one pseudo-block polymer is such that the proportion of the at least one second block with a Tg of greater than or equal to 40° C. ranges from 10% to 85% by weight of the polymer.

34. The aerosol device according to claim 24, wherein the at least one pseudo-block polymer is such that the at least one second block has a Tg of less than or equal to 20° C. and is totally or partially derived from at least one monomer which is such that the homopolymer prepared from the at least one monomer has a glass transition temperature of less than or equal to 20° C.

35. The aerosol device according to claim 24, wherein the at least one pseudo-block polymer is such that the at least one second block has a Tg of less than or equal to 20° C. and is a homopolymer derived from monomers which are such that the homopolymer prepared from these monomers has a glass transition temperature of less than or equal to 20° C.

36. The aerosol device according to claim 34, wherein the at least one pseudo-block polymer is such that the at least one monomer whose corresponding homopolymer has a glass transition temperature of less than or equal to 20° C. is chosen from the following monomers:
acrylates of formula CH₂═CHCOOR₃, wherein R₃ is chosen from linear and branched C₁ to C₁₂ unsubstituted alkyl groups, with the exception of a tert-butyl group, wherein at least one hetero atom chosen from O, N and S is optionally intercalated;
methacrylates of formula CH₂═C(CH₃)—COOR₄, wherein R₄ is chosen from linear and branched C₆ to C₁₂ unsubstituted alkyl groups, wherein at least one hetero atom chosen from O, N and S is optionally intercalated;
vinyl esters of formula R₅—CO—O—CH═CH₂, wherein R₅ is chosen from linear and branched C₄ to C₁₂ alkyl groups;
C₄ to C₁₂ alkyl vinyl ethers; and
N—(C₄ to C₁₂)alkyl acrylamides.

37. The aerosol device according to claim 34, wherein the at least one pseudo-block polymer is such that the at least one monomer whose homopolymer has a glass transition temperature of less than or equal to 20° C. is chosen from alkyl acrylates whose alkyl chain comprises from 1 to 10 carbon atoms, with the exception of a tert-butyl group.

38. The aerosol device according to claim 34, wherein the at least one pseudo-block polymer is such that the proportion of the block with a glass transition temperature of less than or equal to 20° C. ranges from 20% to 90% by weight of the polymer.

39. The aerosol device according to claim 1, wherein the at least one pseudo-block polymer is such that the at least one first block and/or the at least one second block comprises at least one additional monomer.

40. The aerosol device according to claim 39, wherein the at least one pseudo-block polymer is such that the at least one additional monomer is chosen from hydrophilic monomers and ethylenically unsaturated monomers comprising at least one silicon atom.

41. The aerosol device according to claim 39, wherein the at least one pseudo-block polymer is such that the at least one additional monomer is chosen from:
ethylenically unsaturated monomers comprising at least one functional group chosen from carboxylic and sulfonic acid functional groups;
methacrylates of formula CH₂═C(CH₃)—COOR₆ wherein R₆ is chosen from linear and branched alkyl groups comprising from 1 to 4 carbon atoms, wherein the alkyl group is substituted with at least one substituent chosen from hydroxyl groups and halogen atoms;
methacrylates of formula CH₂═C(CH₃)—COOR₉, wherein R₉ is chosen from linear and branched C₆ to C₁₂ alkyl groups wherein at least one hetero atom chosen from O, N and S is optionally intercalated, wherein the alkyl groups are substituted with at least one substituent chosen from hydroxyl groups and halogen atoms;
acrylates of formula CH₂═CHCOOR₁₀, wherein R₁₀ is chosen from linear and branched C₁ to C₁₂ alkyl groups substituted with at least one substituent chosen from hydroxyl groups and halogen atoms, or R₁₀ is chosen from C₁ to C₁₂ alkyl-O—POE (polyoxyethylene) with repetition of the oxyethylene unit 5 to 30 times, or R₁₀ is chosen from polyoxyethylenated groups comprising from 5 to 30 ethylene oxide units.

42. The aerosol device according to claim 31, wherein the at least one pseudo-block polymer is such that the at least one additional monomer is chosen from acrylic acid, methacrylic acid, and trifluoroethyl methacrylate.

43. The aerosol device according to claim 32, wherein the at least one pseudo-block polymer is such that the at least one additional monomer is present in an amount ranging from 1% to 30% by weight, relative to the total weight of the at least one first and/or at least one second blocks.

44. The aerosol device according to claim 1, wherein the at least one pseudo-block polymer is such that each of the at least one first and at least one second blocks comprises at least one monomer chosen from (meth)acrylic acid esters, and optionally at least one monomer chosen from (meth)acrylic acid.

45. The aerosol device according to claim 1, wherein each of the at least one first and at least one second blocks is totally derived from at least one monomer chosen from acrylic acid, (meth)acrylic acid esters and optionally from at least one monomer chosen from (meth)acrylic acid.

46. The aerosol device according to claim 1, wherein the at least one pseudo-block polymer is such that the difference between the glass transition temperatures (Tg) of the at least one first and the at least one second block is greater than 10° C.

47. The aerosol device according to claim 1, wherein the at least one pseudo-block polymer is such that the intermediate segment has a glass transition temperature that is between the glass transition temperatures of the at least one first and at least one second blocks.

48. The aerosol device according to claim 1, wherein the at least one pseudo-block polymer is such that it has a polydispersity index (I) of greater than or equal to 2.5.

49. The aerosol device according to claim 1, wherein the at least one pseudo-block polymer has a polydispersity index ranging from 2.8 to 6.

50. The aerosol device according to claim 1, wherein the weight-average mass (Mw) of the at least one pseudo-block polymer is less than or equal to 300,000.

51. The aerosol device according to claim 1, wherein the weight-average mass (Mw) of the at least one pseudo-block polymer ranges from 35,000 to 200,000.

52. The aerosol device according to claim 1, wherein the number-average mass (Mn) of the at least one pseudo-block polymer is less than or equal to 70,000.

53. The aerosol device according to claim 1, wherein the number-average mass (Mn) of the at least one pseudo-block polymer ranges from 10,000 to 60,000.

54. The aerosol device according to claim 1, wherein the at least one pseudo-block polymer is insoluble in water or in a mixture of water and at least one organic solvent chosen from linear and branched lower monoalcohols comprising from 2 to 5 carbon atoms, without pH modification, at an active material content of at least 1% by weight, at room temperature (25° C.).

55. The aerosol device according to claim 1, wherein the at least one pseudo-block polymer is not an elastomer.

56. The aerosol device according to claim 1, wherein the at least one pseudo-block polymer is chosen from film-forming linear block ethylenic polymers.

57. The aerosol device according to claim 1, wherein the at least one pseudo-block polymer is present in an amount ranging from 0.1% to 60% by weight, relative to the total weight of the at least one styling composition.

58. The aerosol device according to claim 1, wherein the at least one additional fixing polymer is chosen from anionic, cationic, amphoteric and nonionic fixing polymers.

59. The aerosol device according to claim 58, wherein the cationic fixing polymers are chosen from acrylic or methacrylic ester or amide homopolymers and copolymers comprising amino functional groups, cationic polysaccharides, quaternary copolymers of vinylpyrrolidone and vinylimidazole, and chitosans.

60. The aerosol device according to claim 58, wherein the anionic fixing polymers are chosen from acrylic and methacrylic acid homopolymers and copolymers and salts thereof, crotonic acid copolymers, C₄-C₈ monounsaturated carboxylic acid and anhydride copolymers, polyacrylamides comprising carboxylate groups, homopolymers and copolymers comprising sulfonic groups, anionic polyurethanes, and anionic grafted silicone polymers.

61. The aerosol device according to claim 58, wherein the amphoteric fixing polymers are chosen from copolymers comprising acidic vinyl units and basic vinyl units, crosslinked and acylated polyamino amides, polymers comprising zwitterionic units, chitosan-based polymers, modified (C₁-C₅)alkyl vinyl ether/maleic anhydride copolymers, amphoteric polyurethanes, and amphoteric grafted silicone polymers.

62. The aerosol device according to claim 58, wherein the nonionic fixing polymers are chosen from polyalkyloxazolines, vinyl acetate homopolymers and copolymers, acrylic ester homopolymers and copolymers, acrylonitrile copolymers, styrene homopolymers and copolymers, polyamides, vinyllactam homopolymers other than vinylpyrrolidone homopolymers, vinyllactam copolymers, and nonionic grafted silicone polymers.

63. The aerosol device according to claim 58, wherein the at least one additional fixing polymer is chosen from nonionic, anionic, cationic and amphoteric polyurethanes.

64. The aerosol device according to claim 1, wherein the at least one additional fixing polymer is present in an amount ranging from 0.1% to 20% by weight, relative to the total weight of the styling composition.

65. The aerosol device according to claim 1, further comprising at least one thickener.

66. The aerosol device according to claim 65, wherein the at least one thickener is chosen from associative thickening polymers.

67. The aerosol device according to claim 65, wherein the at least one thickener is chosen from gelling agents.

68. The aerosol device according to claim 65, wherein the at least one thickener is present in an amount ranging from 0.01% to 10% by weight, relative to the total weight of the styling composition.

69. The aerosol device according to claim 1, further comprising at least one surfactant.

70. The aerosol device according to claim 69, wherein the at least one surfactant is present in an amount ranging from 0.01% to 40% by weight, relative to the total weight of the composition.

71. The aerosol device according to claim 1, wherein the device is provided in a form chosen from one-compartment devices and two-compartment devices.

72. The aerosol device according to claim 1, wherein the device is chosen from one-compartment devices and the at least one propellent is chosen from liquefied and compressed gases.

73. The aerosol device according to claim 71, wherein the at least one propellant is present in an amount ranging from 6% to 70% by weight, relative to the total weight of materials comprised in the device.

74. A cosmetic treatment process for shaping and/or holding the hairstyle comprising vaporizing onto the hair of the contents of an aerosol device comprising,
at least one propellant and
at least one styling composition comprising, in a cosmetically acceptable medium comprising more than 50% by weight of water relative to the total weight of the composition, at least one pseudo-block polymer and at least one additional fixing polymer, wherein the at least one pseudo-block polymer comprises at least one first block and at least one second block that are mutually incompatible and that have different glass transition temperatures (Tg), wherein the at least one first and at least one second block are linked together via an intermediate segment comprising at least one constituent monomer of the at least one first block and at least one constituent monomer of the at least one second block and wherein the at least one pseudo-block polymer has a polydispersity index (I) of greater than 2.

75. A method for shaping hair, which is persistent over time, comprising vaporizing onto the hair of the contents of an aerosol device comprising,
at least one propellant and
at least one styling composition comprising, in a cosmetically acceptable medium comprising more than 50% by weight of water relative to the total weight of the composition, at least one pseudo-block polymer and at least one additional fixing polymer, wherein the at least one pseudo-block polymer comprises at least one first block and at least one second block that are mutually incompatible and that have different glass transition temperatures (Tg), wherein the at least one first and at least one second block are linked together via an intermediate segment comprising at least one constituent monomer of the at least one first block and at least one constituent monomer of the at least one second block and wherein the at least one pseudo-block polymer has a polydispersity index (I) of greater than 2.

76. A method for shaping the hair, which is moisture-resistant, comprising vaporizing onto the hair of the contents of an aerosol device comprising,
at least one propellant and
at least one styling composition comprising, in a cosmetically acceptable medium comprising more than 50% by weight of water relative to the total weight of the composition, at least one pseudo-block polymer and at least one additional fixing polymer, wherein the at least one pseudo-block polymer comprises at least one first block and at least one second block that are mutually incompatible and that have different glass transition temperatures (Tg), wherein the at least one first and at least one second blocks are linked together via an intermediate segment comprising at least one constituent monomer of the at least one first block and at least one constituent monomer of the at least one second block and wherein the at least one pseudo-block polymer has a polydispersity index (I) of greater than 2.
